(19) United States
(12) Patent Application Publication
(10) Pub. No.: US 2023/0172336 A1
(43) Pub. Date: Jun. 8, 2023

(54) ULTRASONIC RADIATION APPARATUS AND HAIR CARE APPARATUS

(71) Applicant: Pixie Dust Technologies, Inc., Tokyo (JP)

(72) Inventors: Masato KOJIMA, Tokyo (JP); Daiki SHIMO, Tokyo (JP); Yoshiki OOKA, Tokyo (JP)

(73) Assignee: Pixie Dust Technologies, Inc., Tokyo (JP)

(21) Appl. No.: 18/163,037

(22) Filed: Feb. 1, 2023

Related U.S. Application Data
(63) Continuation of application No. PCT/JP2022/020340, filed on May 16, 2022.

(30) Foreign Application Priority Data
Nov. 15, 2021 (JP) 2021-185973
Mar. 1, 2022 (JP) 2022-031125

(51) Int. Cl. A45D 20/14; F26B 3/28

(52) U.S. Cl. CPC A45D 20/14; F26B 3/28

(57) ABSTRACT

The ultrasonic radiation apparatus includes a first transducer support having a substantially planar first main face. The first transducer support has a plurality of first mounts on the first main face. The apparatus includes a second transducer support having a substantially planar second main face. The second transducer support has a plurality of second mounts on the second main face, and is arranged such that a second normal of the second main face intersects a first normal of the first main face at a common target point. The apparatus includes a plurality of first ultrasonic waves attached to the plurality of first mounts to radiate ultrasonic waves traveling along the first normal. The apparatus includes a plurality of second ultrasonic transducers attached to the plurality of second mounts to radiate ultrasonic waves traveling along the second normal.

### CROSS-REFERENCE TO RELATED APPLICATION

This application is a Continuation Application of No. PCT/JP2022/20340, filed on May 16, 2022, and the PCT application is based upon and claims the benefit of priority from Japanese Patent Application No. 2021-185973, filed on Nov. 15, 2021, and Japanese Patent Application No. 2022-031125, filed on Mar. 1, 2022, the entire contents of which are incorporated herein by reference.

### TECHNICAL FIELD

The present disclosure relates to ultrasonic radiation apparatus and hair care apparatus.

### BACKGROUND

In general, people with head hair problems (e.g., thinning or graying hair) take various measures for hair care. For example, by promoting hair growth or hair regrowth, head hair problems (thinning or hair loss) are eliminated. Various means of promoting hair growth or hair regrowth are known.

For example, Japanese Patent Application Publication No. 2015-134144 discloses a hair growth and hair regrowth device that promotes hair growth or hair regrowth by attaching electrodes to the scalp of the subject.

However, there are concerns about adverse effects on hygiene in hair care, since the technology in the Japanese Patent Application Publication No. 2015-134144 brings a part of the hair-growth device into contact with the scalp.

### BRIEF DESCRIPTION OF DRAWINGS

FIG. 1 is a block diagram showing the configuration of hair care apparatus according to a present embodiment.

FIG. 2 shows the appearance of the ultrasonic radiation apparatus according to the present embodiment.

FIG. 3 shows the appearance of the ultrasonic radiation apparatus according to the present embodiment.

FIG. 4 shows the appearance of the transducer support provided by the ultrasonic radiation apparatus according to the present embodiment.

FIG. 5 shows the state in which a plurality of ultrasonic transducers are attached to the transducer support provided by the ultrasonic radiation apparatus according to the present embodiment.

FIG. 6 is a block diagram showing the configuration of the controller according to the present embodiment.

FIG. 7 is an illustration of one aspect of the present embodiment.

FIG. 8 is an illustration of the focusing of ultrasonic waves radiated by the ultrasonic radiation apparatus according to the present embodiment.

FIG. 9 is a graph showing the time variation of the amplitude of ultrasonic waves radiated by the ultrasonic transducers according to the present embodiment.

FIG. 10 is a flowchart of the control process according to the present embodiment.

FIG. 11 shows a first example of the external shape of the hair care apparatus according to the present embodiment.

FIG. 12A shows a second example of the external shape of the hair care apparatus according to the present embodiment.

FIG. 12B shows a second example of the external shape of the hair care apparatus according to the present embodiment.

FIG. 13 shows a third example of the external shape of the hair care apparatus according to the present embodiment.

FIG. 14 is a front view of a fourth example of the external shape of the hair care apparatus according to the present embodiment.

FIG. 15 is a side view of the fourth example of the external shape of the hair care apparatus according to the present embodiment.

FIG. 16 is an example of a head attachment.

FIG. 17 is an example of a head attachment.

FIG. 18 shows a fifth example of the external shape of the hair care apparatus according to the present embodiment.

FIG. 19 is a block diagram showing the configuration of the hair care apparatus in Modification 1.

FIG. 20 is a side view of the hair care apparatus in Modification 2.

FIG. 21 is a front view of the hair care apparatus in Modification 2.

FIG. 22 is an illustration of one aspect of Modification 3.

FIG. 23 is a front view of the hair care apparatus in Modification 4.

FIG. 24 is a front view of the hair care apparatus in Modification 5.

FIG. 25 shows the appearance of the ultrasonic radiation apparatus in Modification 6.

FIG. 26 shows the appearance of the ultrasonic radiation apparatus in Modification 6.

FIG. 27 is an illustration of the target point of the side and bottom supports provided by the ultrasonic radiation apparatus in Modification 6.

FIG. 28 shows the state in which a plurality of ultrasonic transducers are attached to the side supports of Modification 6.

FIG. 29 shows the state in which a plurality of ultrasonic transducers are attached to the bottom support in Modification 6.

FIG. 30 shows the state in which a plurality of ultrasonic transducers are attached to the bottom support in Modification 6.

FIG. 31 shows the appearance of the attachment in Modification 6.

### DETAILED DESCRIPTION

Hereinafter, an embodiment of the present disclosure is described in detail based on the drawings. Note that, in the drawings for describing the embodiments, the same components are denoted by the same reference sign in principle, and the repetitive description thereof is omitted.

An ultrasonic radiation apparatus of an aspect of the present disclosure comprises:

a first transducer support with a substantially planar first main face, the first transducer support having a plurality of first mounts on the first main face;

a second transducer support with a substantially planar second main face, the second transducer support having a plurality of second mounts on the second main face and being arranged such that a second normal of the second main face intersects a first normal of the first main face at a common target point;

a plurality of first ultrasonic transducers attached to the plurality of first mounts to radiate ultrasonic waves traveling along the first normal; and

a plurality of second ultrasonic transducers attached to the plurality of second mounts to radiate ultrasonic waves traveling along the second normal.

In subsequent descriptions, a common reference sign, such as “99,” may be used when describing a common explanation for several similar elements. On the other hand, when describing these elements individually, a common reference sign with subscripts, such as “99-1” or “99 2”, etc., may be used to describe each of these elements individually.

Unless otherwise noted in the descriptions that follow, the various orientations are defined with respect to the ultrasonic radiation apparatus.

The direction forward of the ultrasonic radiation apparatus (i.e., the direction in which the composite component of the ultrasonic waves radiated by the plurality of ultrasonic transducers in the ultrasonic radiation apparatus travels) is defined as the Z+ direction, and the direction behind the ultrasonic radiation apparatus, i.e., the direction opposite the Z+ direction, is defined as the Z -direction.

The left, right, upper, and lower directions of the ultrasonic radiation apparatus are defined as X+, X−, Y+, and Y− directions, respectively.

The X, Y, and Z axes are orthogonal to each other.

The direction of travel of each ultrasonic wave radiated by the multiple ultrasonic transducers all include a Z+ component. Each ultrasound may further include at least one of the X+, X−, Y+, and Y− components.

### (1) Configuration of Hair Care Apparatus

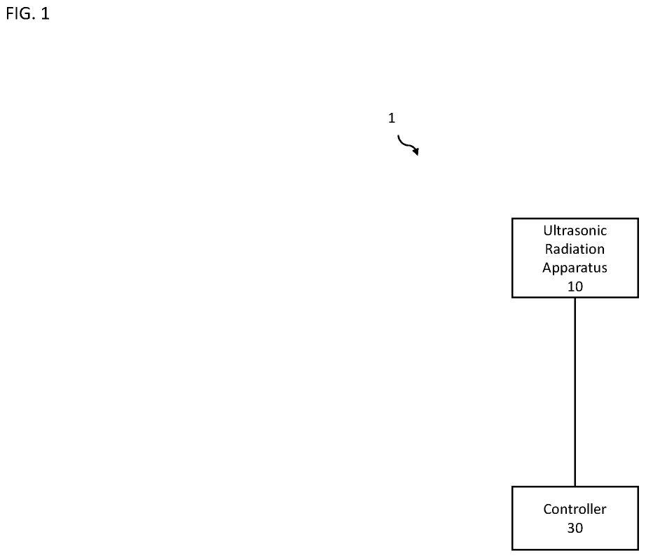

The configuration of the hair care apparatus is described. FIG. 1 is a block diagram of the hair care apparatus according to the present embodiment.

As shown in FIG. 1, the hair care apparatus 1 is equipped with an ultrasonic radiation apparatus 10 and a controller 30.

The ultrasonic radiation apparatus 10 and the controller 30 are connected via a predetermined interface (e.g., a communication cable or a wireless ((e.g., Bluetooth (registered trademark) or WiFi) channels) that can transmit electrical signals (e.g., drive signals (an example of a control signal) as described below).

The ultrasonic radiation apparatus 10 is configured to radiate ultrasound waves according to control by the controller 30.

Controller 30 is configured to control ultrasonic radiation apparatus 10. Controller 30 is an example of an information processing apparatus.

### (1-1) Configuration of Ultrasonic Radiation Apparatus

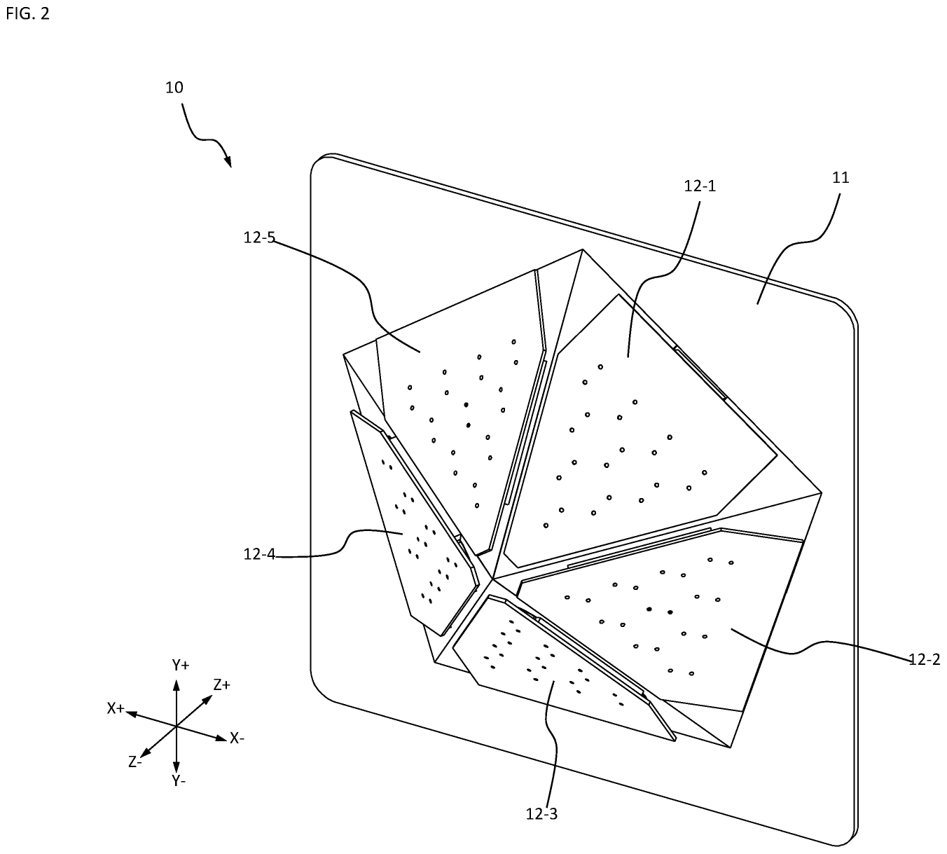

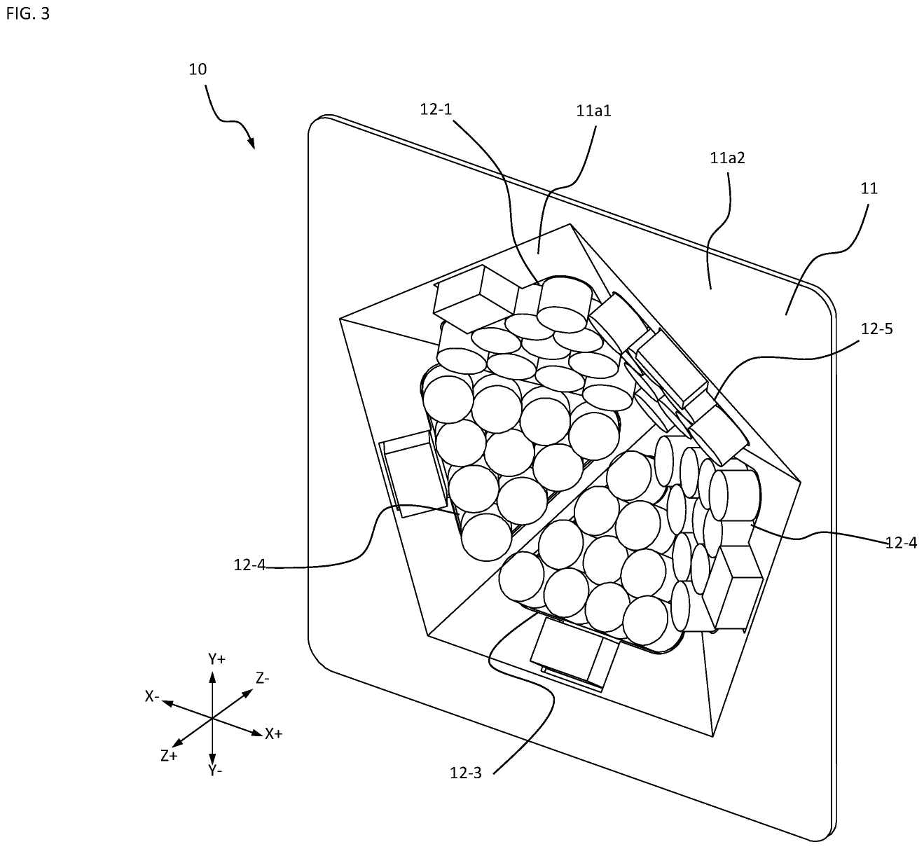

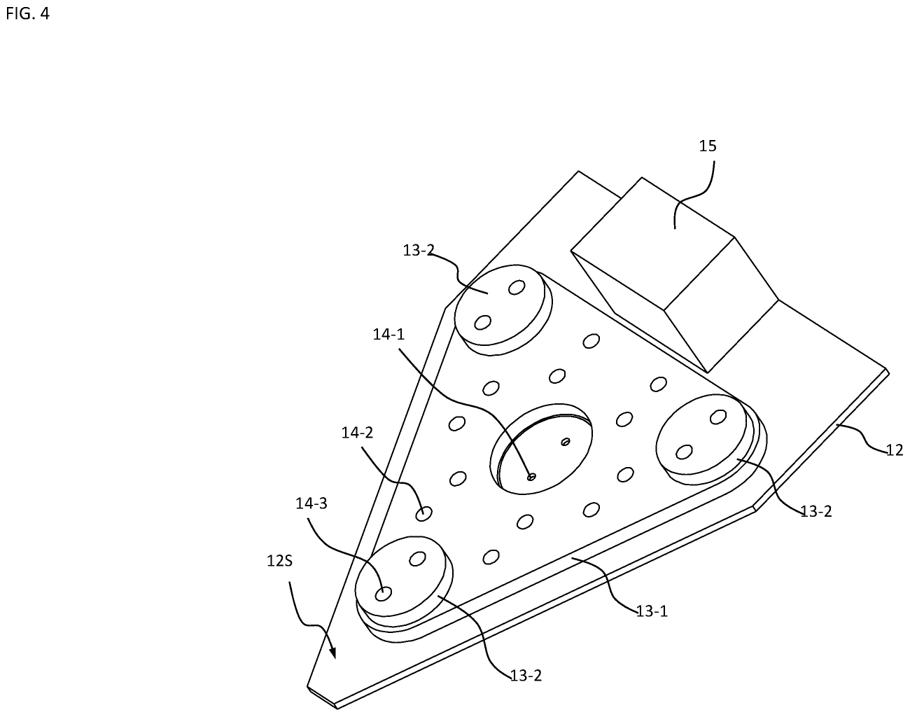

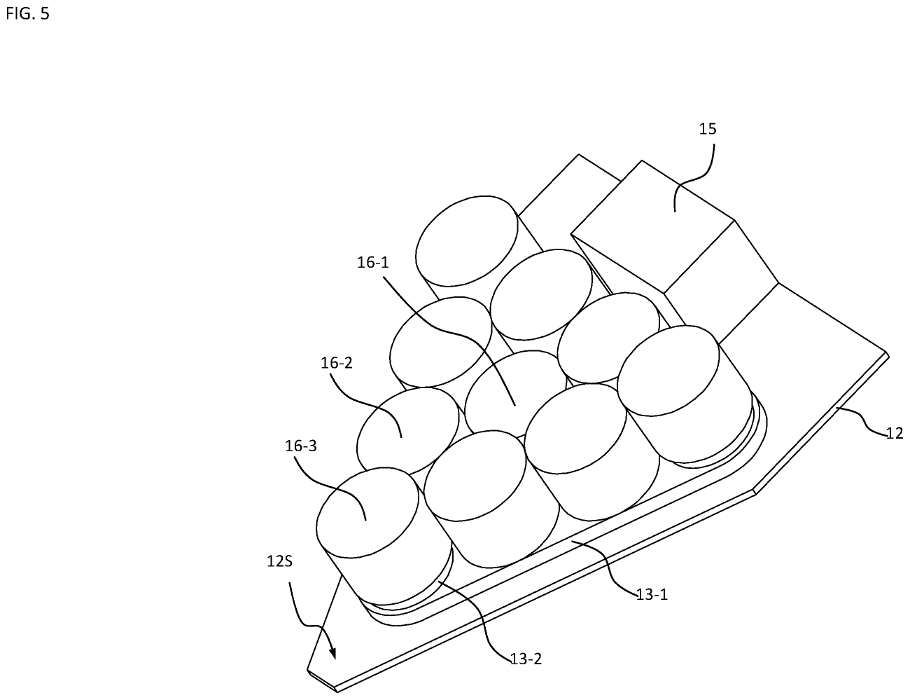

The configuration of the ultrasonic radiation apparatus is described below. FIG. 2 shows the appearance of the ultrasonic radiation apparatus according to the present embodiment. FIG. 3 shows the appearance of the ultrasonic radiation apparatus according to the present embodiment. FIG. 4 shows the appearance of the transducer support provided by the ultrasonic radiation apparatus according to the present embodiment. FIG. 5 shows the state in which a plurality of ultrasonic transducers are attached to the transducer support provided by the ultrasonic radiation apparatus according to the present embodiment.

As shown in FIGS. 2 and 3, the ultrasonic radiation apparatus 10 has a base 11, five transducer supports 12-1, 12-2, 12-3, 12-4, 12-5. As shown in FIGS. 4 and 5, the ultrasonic radiation apparatus 10 has, for each transducer support 12, a first attachment 13-1, a plurality of second attachments 13-2, a plurality of mounts 14-1, 14-2, 14-3, a connector 15, and a plurality of ultrasonic transducers 16-1, 16-2, 16-3.

The base 11 includes a first region 11a1 and a second region 11a2. The second region 11a2 is a substantially planar face, for example. In the first region 11a1 a plurality of polygonal faces that differ from each other in at least one of their positions and orientations are combined. The combination of the multiple polygonal faces constitutes a three-dimensional shape that is convex in the Z− direction. A plurality of polygonal faces are combined so that the normal extending from each face to the direction of ultrasound travel intersects one of the normals extending from the other faces to the direction of ultrasound travel at a common point of intersection. As an example, as shown in FIGS. 2 and 3, in the first region 11a1, five substantially equilateral triangular faces sharing one vertex of an icosahedron are combined. The five substantially equilateral triangular faces have an umbrella-like three-dimensional shape convex in the Z− direction. The second region 11a2 surrounds the first region 11a1.

The base 11 is provided with means (e.g., screw holes) for mounting each of the transducer supports 12 (an example of “first transducer support” and “second transducer support”) on each face comprising the first area 11a1. The base 11 is configured to support the transducer supports 12 with the transducer supports 12 attached to base 11.

The base 11 has means (e.g., screw holes) for attachment to an object not shown (e.g., the housing of the hair care apparatus 1), e.g., in the second region 11a2. With the base 11 attached to an object, the base 11 can maintain (i.e., stand still) its position and orientation (e.g., the angle of rotation around the X, Y, and Z-axis) with respect to that object can be maintained (i.e., stationary).

As shown in FIGS. 3 and 4, the transducer support 12 has a substantially planar main face 12S (an example of “first main face” and “second main face”) and is attached to each of the faces of the first region 11a1 of the base 11 so that the main face 12S faces in the direction of the ultrasonic wave. The transducer support 12 is provided with means (e.g., screw holes) for attaching the transducer support 12 to the base 11. With the transducer support 12 attached to the base 11, the transducer support 12 can maintain its position and orientation (i.e., remain stationary) relative to said base 11. Specifically, the transducer support 12 is mounted on the base 11 so that one of the normals extending from the main face 12S to the direction of ultrasonic wave travel (an example of “first normal” and “second normal”) intersects at a common target point with one of the normals extending from the main face 12S of another transducer support 12 to the direction of ultrasonic wave travel. The target point may coincide with or differ from the aforementioned common point of intersection. The aforementioned common point of intersection coincides with the target point when the main face 12S of the transducer support 12 is parallel to the face of the first region 11a1 to which said transducer support 12 is attached. Furthermore, each of the transducer supports 12 may be mounted on the base 11 so that the distance from the main face 12S of the transducer support 12 to the aforementioned target point is substantially equal to each other

The main face 12S has the same or similar shape as the face comprising the first region 11a1of the base 11. As an example, the main face 12S is shaped like an equilateral triangle.

As shown in FIG. 4, the transducer support 12 has a plurality of mounts 14-1, 14-2, 14-3 on the main face 12S. The mount 14 (an example of a “first mount” or “second mount”) is configured to allow mounting the ultrasonic transducer 16 (an example of “first ultrasonic transducer” or “second ultrasonic transducer”). With the ultrasonic transducer 16 attached to the mount 14, the transducer support 12 is configured to support the ultrasonic transducer 16 and to provide electrical connection to the connector 15.

More precisely, as shown in FIG. 4, on the main face 12S of the transducer support 12, there is a first attachment 13-1, and a plurality of second attachments 13-2 are attached. The first attachment 13-1 and the plurality of second attachments 13 -2 are configured to allow adjustment of the distance between at least a part of the ultrasonic transducers 16 and the main face 12S. Specifically, the first attachment 13-1 and the plurality of second attachments 13-2 form a stepped staircase-like area on the main face 12S with respect to a direction orthogonal to the main face 12S. As an example, the staircase formed by the first attachment 13-1 and the plurality of second attachments 13-2 is, for example, shaped such that the height in the center is the lowest (i.e., the distance from the main surface 12S is small) and the height increases away from the center toward the periphery (i.e., the distance from the main surface 12S increases).

As shown in FIG. 5, an ultrasonic transducer 16-1 can be directly mounted on the mount 14-1. The ultrasonic transducer 16-2 can be attached to the mount 14-2 via the first attachment 13-1. The ultrasonic transducer 16-3 can be attached to the mount 14-3 via the first attachment 13-1 and the second attachment 13-2. Therefore, the distance between the ultrasonic transducer 16-1 and the main face 12S is smaller than the distance between the ultrasonic transducer 16-2 and the main face 12S. The distance between the ultrasonic transducer 16-2 and the main face 12S is smaller than the distance between the ultrasonic transducer 16-3 and the main face 12S.

Therefore, the distance from each ultrasonic transducer 16 to the main face 12S is not uniform. In other words, each ultrasonic transducer 16 mounted on the transducer support 12 is mounted at a different distance from the main face 12S than at least one of the other ultrasonic transducers 16. As an example, each of the plurality of ultrasonic transducers 16 is mounted at an offset position such that the greater the distance from the reference point on the main face 12S (e.g., the position of the mount 14-1) to the mount 14 corresponding to the ultrasonic transducer 16, the greater the distance from the main surface 12S with respect to a direction orthogonal to the main face 12. Specifically, the attachment position of each ultrasonic transducer 16 can be determined as a position on a sphere centered at a point a predetermined distance away from the main face 12S. In other words, the attachment positions of the plurality of ultrasonic transducers 16 can be defined so that the distances from a point a predetermined distance away from the main face 12S are substantially equal to each other.

As shown in FIGS. 4 and 5, the transducer support 12 supports a connector 15 on the main face 12S. The connector 15 is connected to an external device (e.g., controller 30) to enable the exchange of electrical signals.

With the ultrasonic transducer 16 attached (directly or indirectly via attachment 13-1, or attachment 13-1 and attachment 13-2) to the mount 14, the ultrasonic transducer 16 can maintain its position and orientation (i.e., remain stationary) relative to the transducer support 12. The ultrasonic transducer 16 is electrically connected to the connector 15 when the ultrasonic transducer is attached to the mount 14. The ultrasonic transducer 16 is configured to receive the drive signal transmitted by the controller 30 via the connector 15 and radiate ultrasonic wave according to the drive signal.

(1-2) Configuration of controller

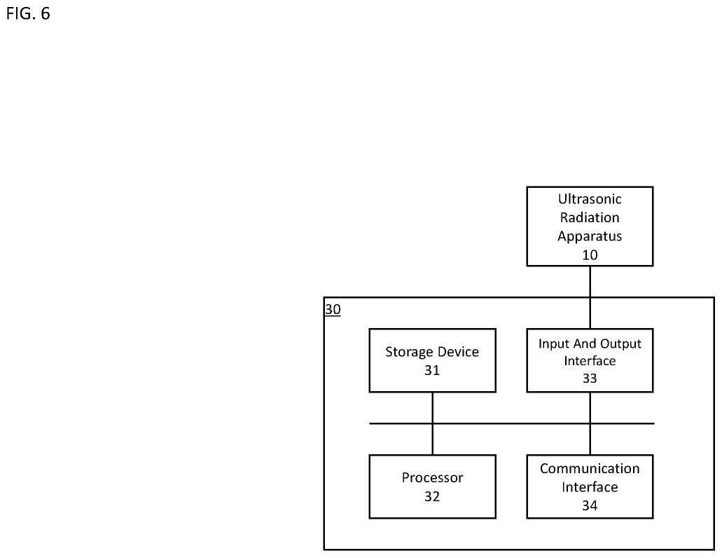

The configuration of the controller is described below. FIG. 6 is a block diagram showing the configuration of the controller according to the present embodiment. As shown in FIG. 6, controller 30 has a storage device 31, processor 32, input/output interface 33, and a communication interface 34.

The storage device 31 is configured to store a program and data. The storage device 31 is, for example, a combination of a ROM (read only memory), a RAM (random access memory), and a storage (for example, a flash memory or a hard disk).

Programs include, for example, the following programs:

operating System (OS) programs; and

application programs for performing information processing (e.g., control programs for ultrasonic radiation apparatus 10).

The data includes, for example, the following data:

databases referenced in information processing

data obtained by executing an information processing (that is, an execution result of an information processing)

The processor 32 is a computer that realizes the functions of the controller 30 by activating a program stored in the memory device 31. Processor 32 is, for example, at least one of the following:

CPU (Central Processing Unit);

GPU (Graphic Processing Unit);

ASIC (Application Specific Integrated Circuit); and

FPGA (Field Programmable Array).

The input/output interface 33 is configured to obtain information (e.g., user instructions) from an input device connected to the controller 30 and to output information (e.g., control signals) to an output device connected to the controller 30.

Input devices are, for example, physical switches, sensors, keyboards, pointing devices, touch panels, or combinations thereof.

The output device is, for example, an ultrasonic radiation apparatus 10, a display, a speaker, or a combination thereof.

The communication interface 34 is configured to control communication between the controller 30 and an external device (e.g., a web server or client device (as an example, a smartphone, tablet device, or personal computer)).

### (2) One Aspect of Embodiment

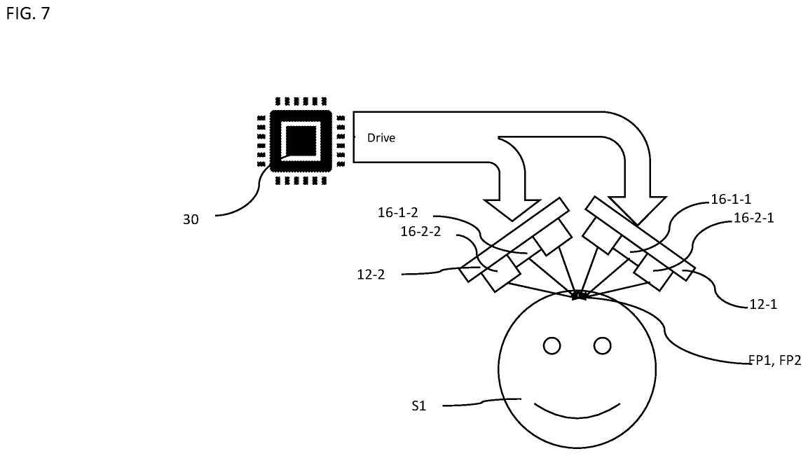

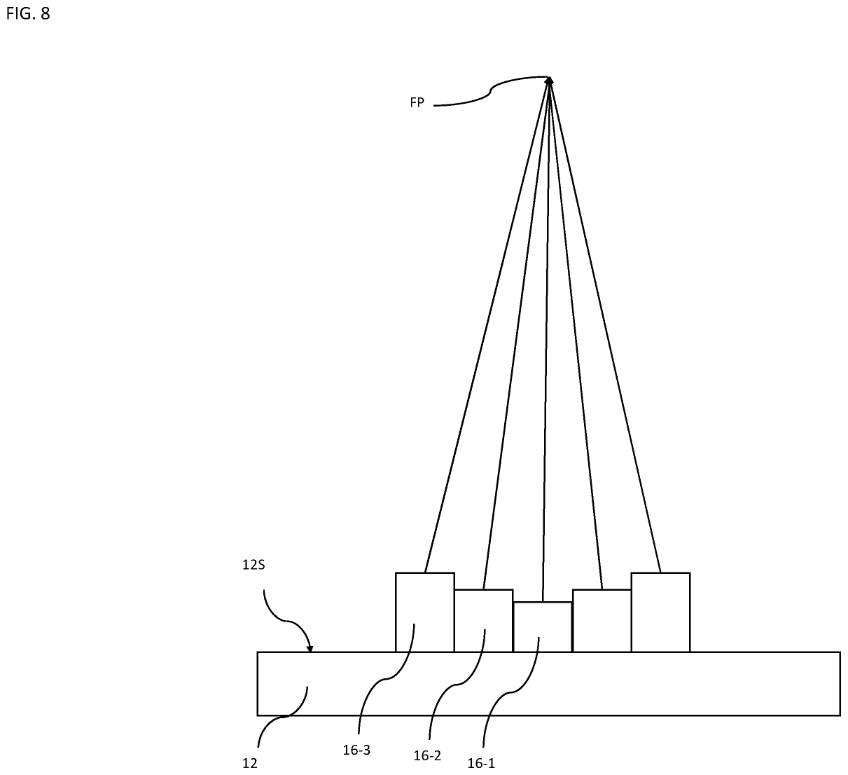

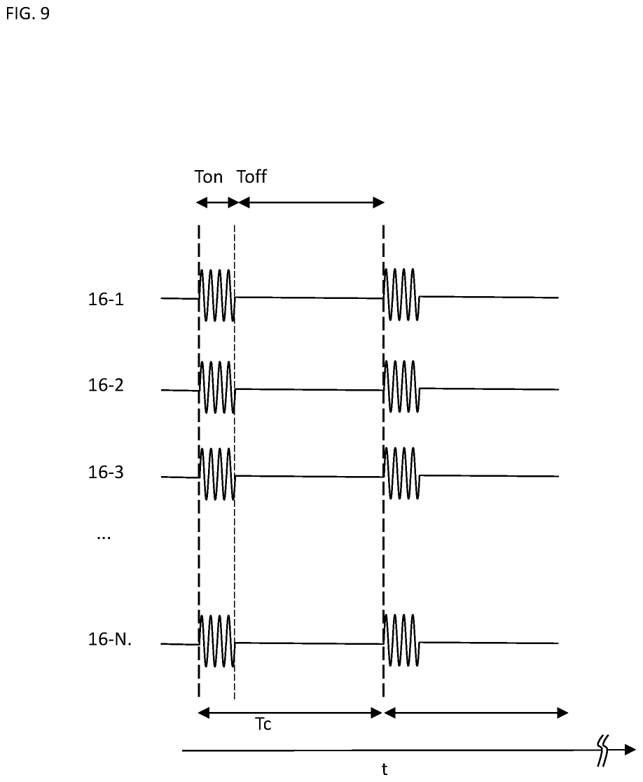

This section describes one aspect of the present embodiment. FIG. 7 is an illustration of one aspect of the present embodiment. FIG. 8 illustrates the focusing of ultrasonic waves radiated by the ultrasonic radiation apparatus according to the present embodiment. FIG. 9 is a graph showing the time variation of the amplitude of ultrasonic waves radiated by the ultrasonic transducers according to the present embodiment.

As shown in FIG. 7, during operation of the hair care apparatus 1, the controller 30 (processor 32 in detail) generates a drive signal and transmits the signal to the ultrasonic radiation apparatus 10. Each connector 15 receives a drive signal and transmits (relays) the drive signal to each ultrasonic transducer 16 connected to the connector 15. Controller 30 generates one common drive signal for the ultrasonic radiation apparatus 10. In other words, if the transmission time difference of the drive signal is ignored, each ultrasonic transducer 16 radiates ultrasonic waves at substantially the same time.

As shown in FIG. 8, a plurality of ultrasonic transducers 16 mounted on a certain main face 12S are mounted such that their distances from the point FP, which is a predetermined distance away from the main face 12S, are substantially equal. Therefore, when each ultrasonic transducer 16 radiates ultrasonic waves (actually corresponding to ultrasonic beams or focused ultrasonic waves, but simply referred to as “ultrasonic waves”) at the same time, the ultrasonic waves are in phase at the point FP and intensify each other at point FP. Therefore, the point FP is a focal point where ultrasonic waves radiated by a plurality of ultrasonic transducers 16 mounted on a certain main face 12S converge. In other words, the controller 30 can focus the ultrasonic waves radiated by the plurality of ultrasonic transducers 16 mounted on a certain main face 12S to a point FP by driving the plurality of ultrasonic transducers 16 at the same time without adjusting the time difference between them.

Therefore, the ultrasonic waves radiated by the ultrasonic transducers 16-1-1, 16-1-2 attached to the transducer support 12-1 are focused to a point FP1 in a predetermined positional relationship with the main face of the transducer support 12-1. Similarly, ultrasonic waves radiated by ultrasonic transducers 16-2-1, 16-2-2 attached to the transducer support 12-2 are focused to a point FP2 in a predetermined position relative to the main face of the transducer support 12-2. By installing (i.e., mounting on the base 11) the transducer supports 12-1 and 12-2 so that points FP1 and FP2 are coincident, the acoustic radiation pressure applied to the focal point (i.e., points FP1 and FP2) can be further increased.

When the head (e.g., scalp, hair (including hair roots, the same hereinafter), or a combination thereof), which is the treatment area of the subject S1, is located at the ultrasonic wave focal point (i.e., point FP1 or FP2), the ultrasonic waves radiated from the plurality of ultrasonic transducers 16 can apply acoustic radiation pressure to the treatment area. The applied acoustic radiation pressure causes the treatment area of the subject S1 to receive pressure stimulation along the Z+ direction without making contact with any of the 16 ultrasonic transducers (hereinafter referred to as “non-contact vibration pressure stimulation”). Controller 30 drives a plurality of ultrasonic transducers 16 at a predetermined vibration frequency (e.g., 10 Hz) intermittently. Specifically, as shown in FIG. 9, the controller 30 drives the controller 30 controls the plurality of ultrasonic transducers 16 using a pulse signal such that the plurality of ultrasonic transducers 16 are driven over the first period Ton and the plurality of ultrasonic transducers 16 are not driven over the second period Toff. The acoustic radiation pressure that pressed down on the treated area of the subject S1 in the first period Ton is removed in the second period Toff. When the acoustic radiation pressure is removed, the treated area of the subject S1 is restored by the elasticity of the scalp. In other words, the cycle Tc, which includes the first period Ton and the second period Toff are repeated, acoustic radiation pressure is applied intermittently to the treatment area of the subject S1. As a result, the treated area is stimulated by acoustic radiation pressure (hereinafter referred to as “non-contact vibration pressure stimulation”) at the above vibration frequency without making contact with any of the ultrasonic transducers 16. When the scalp of subject S1 is vibrated, the resting hair papilla cells in the head move into the growth phase due to the activation caused by the vibration. Growth of papilla cells during the growth phase is accelerated by the activation caused by vibration. This will have the effect of promoting hair regrowth or hair growth of the subject S1 without adversely affecting the hygiene of the subject S1. In addition, when the scalp of subject S1 vibrates, pigment cells (melanocytes) are activated. This has the effect of improving the gray hair of the subject S1.

As an example, the controller 30 may intermittently apply acoustic radiation pressure to the treatment area the subject S1 by radiating ultrasonic waves modulated at a predetermined vibration frequency from the plurality of ultrasonic transducers 16. An example of modulation is amplitude modulation (AM (Amplitude Modulation)). The controller 30 may provide a PWM (Pulse Width Modulation) modulated drive signal to the plurality of ultrasonic transducers 16, thereby causing said plurality of ultrasonic transducers 16 to radiate amplitude-modulated ultrasonic waves.

According to the ultrasonic radiation apparatus 10, the controller 30 does not need to adjust the drive time difference of the multiple ultrasound transducers 16. This simplifies control compared to a configuration that adjusts the drive time difference of multiple ultrasonic transducers 16 (hereinafter referred to as “ultrasonic phased array”).

According to the ultrasonic radiation apparatus 10, ultrasonic waves radiated from each ultrasonic transducer 16 reach the focal point by the shortest path, thus suppressing distance attenuation of the ultrasonic waves. This makes it more energy efficient than ultrasonic phased arrays.

According to the ultrasonic radiation apparatus 10, the transducer support 12 does not contain curved surfaces. This simplifies assembly and mounting compared to supports that include curved surfaces (e.g., bowl-shaped supports).

### (3) Control Processing

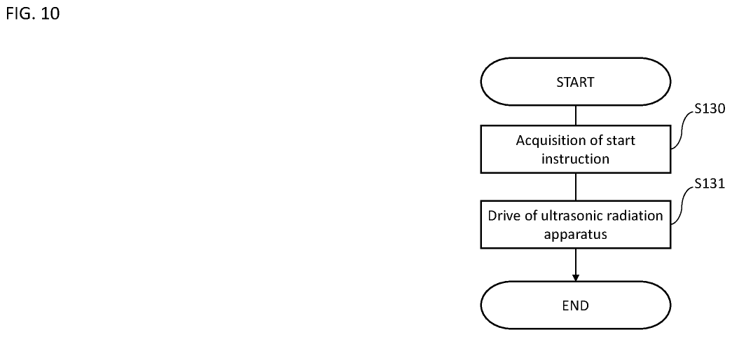

This section describes the control process of the present embodiment. FIG. 10 is a flowchart of the control process according to the present embodiment.

Before the start of the control process in FIG. 10, the subject or practitioner sets up the hair care apparatus 1. The practitioner is the person who provides hair care to the subject using the hair care apparatus 1. The practitioner may be, for example, a person engaged in hair care services or a medical professional. The setting of the hair care apparatus 1 can include, for example, at least one of the following:

powering on the hair care apparatus 1

adjustment of the positional relationship between the ultrasonic radiation apparatus 10 and the treatment area of the subject;

assigning a marker to the treatment area; and

application of hair care agents (including hair packs) to the treatment area.

Here, in the above positional adjustment, the subject or the practitioner may change the position or posture of at least one of the ultrasonic radiation apparatus 10, or the subject.

As shown in FIG. 10, controller 30 executes acquisition of start instruction (S130).

In the first example of acquisition of start instruction (S130), the controller 30 acquires start instructions in response to operations performed on an input device (e.g., physical switch, keyboard, pointing device, touch panel) connected to the controller 30. The subject of the operation may be the person being treated or the practitioner.

In the second example of acquisition of start instruction (S130), the controller 30 acquires a start instruction in accordance with the detection result of the treatment area of the subject by the sensor (e.g., optical sensor) connected to the controller 30. As an example, a marker can be placed at the treatment area to assist the optical sensor in detecting the treatment area.

In the third example of acquisition of start instruction (S130), a start instruction is acquired according to the combination of the operation performed on the input device connected to the controller 30 and the detection result of the treatment area of the subject by the sensor connected to the controller 30.

If the drive and stop periods of ultrasonic transducer 16 (i.e., the first period Ton and second period Toff in FIG. 9) are configured to be variable, the start instruction may include information indicating the length of the periods. The length of the drive and stop periods of the ultrasonic transducers 16 may be specified by the practitioner or the subject, or may be determined by an algorithm.

After step S130, controller 30 executes drive of ultrasonic radiation apparatus (S131).

Specifically, controller 30 generates and transmits a drive signal to ultrasonic radiation apparatus 10. The controller 30 periodically generates control signals to drive the ultrasonic transducers 16 intermittently until the end of the period during which the treatment with the hair care apparatus 1 is performed (hereinafter referred to as the “treatment period”). The treatment period is, for example, 5 minutes.

As an example, the controller 30 repeats the cycle of driving the ultrasonic transducer 16 over a drive period and stopping the ultrasonic transducer 16 over the stop period that follows the drive period. In other words, controller 30 performs pulse control of ultrasonic transducer 16.

The ultrasonic transducer 16 receives the drive signal via the corresponding connector 15. Ultrasonic transducers 16 radiate ultrasonic waves substantially at the same time in response to the received drive signal. Ultrasonic waves radiated by a group of ultrasonic transducers 16 mounted on the transducer support 12 are focused at a focal point in a predetermined positional relationship to the main face 12S of the transducer support 12.

When the treatment period of the hair care apparatus 1 is configured to be variable, the start instruction may include information indicating the length of the treatment period. The length of the treatment period with the hair care apparatus 1 may be specified by the practitioner or the subject, or determined by an algorithm.

With the completion of step S131, controller 30 terminates the control process (FIG. 10).

### (4) External Shape of Hair Care Apparatus

The external shape of the hair care apparatus 1 is described below.

### (4-1) First Example of External Shape of Hair Care Apparatus

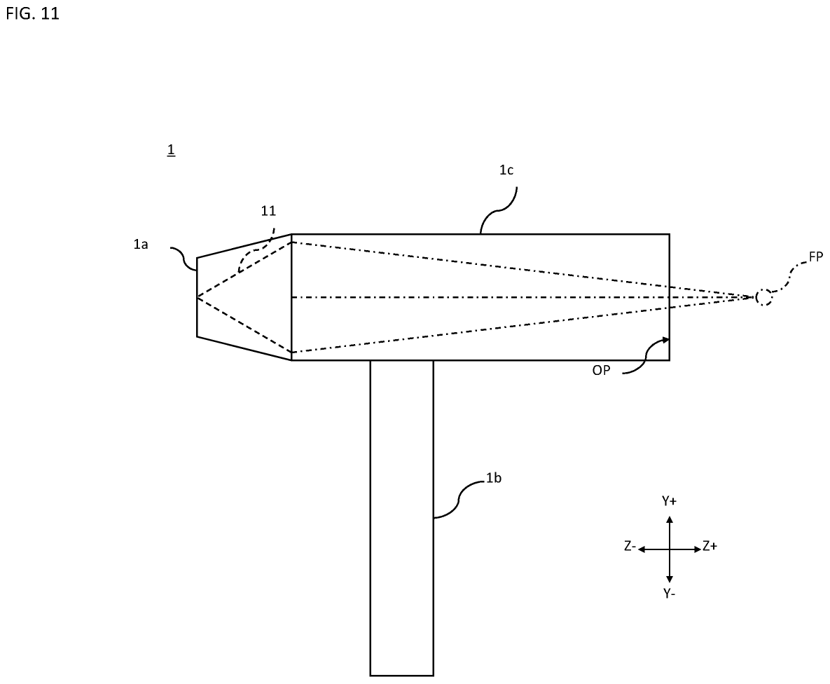

The following is a first example of the external shape of the hair care apparatus 1. FIG. 11 shows a first example of the external shape of the hair care apparatus according to the present embodiment.

As shown in FIG. 11, the hair care apparatus 1 has a base cover 1a, a grip 1b, and a housing 1c.

The housing 1c has an opening OP. The interior of housing 1c is hollow. The housing 1c is connected to the base cover 1a and the grip 1b. The housing 1c extends along the direction of ultrasonic radiation (Z+ direction).

The shape of the base cover 1a is substantially trapezoidal. Base cover 1a covers base 11. Base 11 is positioned where ultrasonic waves are radiated toward the aperture OP (i.e., toward the Z+ direction).

Grip 1b extends substantially orthogonally to housing 1c.

According to the hair care apparatus 1 shown in FIG. 11, the user of the hair care apparatus 1 uses the hair care apparatus 1 while holding the grip 1b and with the point FP positioned on the affected area of the hair care (i.e., with the opening OP facing the affected area). In this case, the user can use the hair care apparatus 1 like a hair dryer.

### (4-2) Second Example of External Shape of Hair Care Apparatus

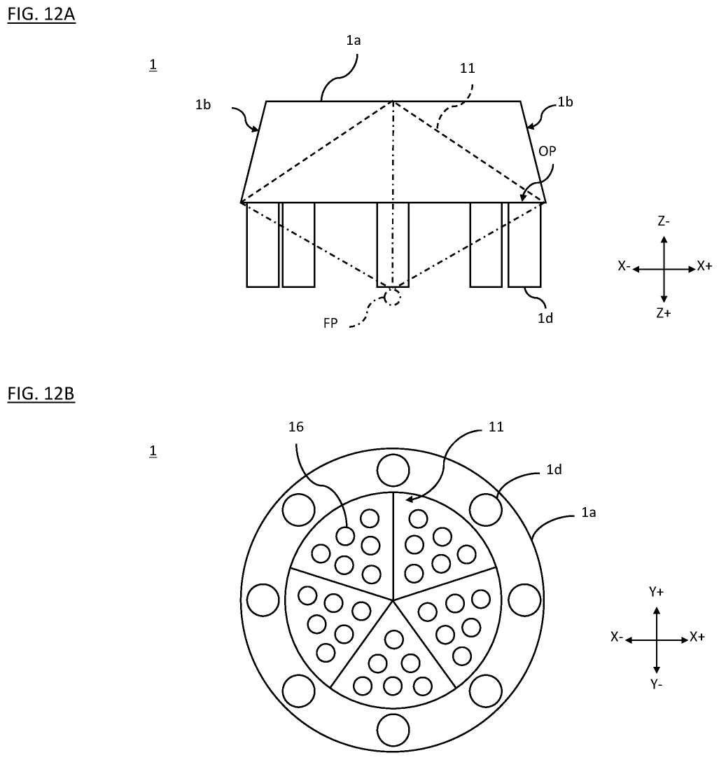

A second example of the external shape of the hair care apparatus 1 is described below. FIG. 12A shows a second example of the external shape of the hair care apparatus according to the present embodiment. FIG. 12B shows a second example of the external shape of the hair care apparatus according to the present embodiment.

As shown in FIGS. 12A and 12B, the hair care apparatus 1 has a base cover 1a and a guide 1d.

The base cover 1a has an opening OP. The base cover 1a is substantially trapezoidal in shape. Base cover 1a covers base 11. The base 11 is positioned where ultrasonic waves are radiated toward the aperture OP (i.e., in the Z+ direction).

Base cover 1a has grip 1b. The grip 1b is the part that the hand of the user using the hair care apparatus 1 contacts. The grip 1b is formed of at least one of the following: a shape that is easy for the user to grip the hair care apparatus 1 and a slip-resistant material. Guide 1d is located at the Z+ end of base cover la. The length of guide 1d in the Z direction roughly corresponds to the length in the Z direction from the end of the base cover 1a (i.e., the opening OP) to the point FP. The guide 1d is made of a material (e.g., silicone) that applies moderate pressure to the scalp.

According to the hair care apparatus 1 shown in FIGS. 12A and 12B, the user of the hair care apparatus 1 uses the hair care apparatus 1 while holding the grip 1b and with the guide 1d in contact with the scalp. In this case, the user can use the hair care apparatus 1 as a scalp massager.

Guide 1d allows the user to use the hair care apparatus 1 while keeping the distance from the Z+ end of the base cover 1a (i.e., the opening OP) to the point FP and the distance from the end to the affected area.

### (4-3) Third Example of External Shape of Hair Care Apparatus

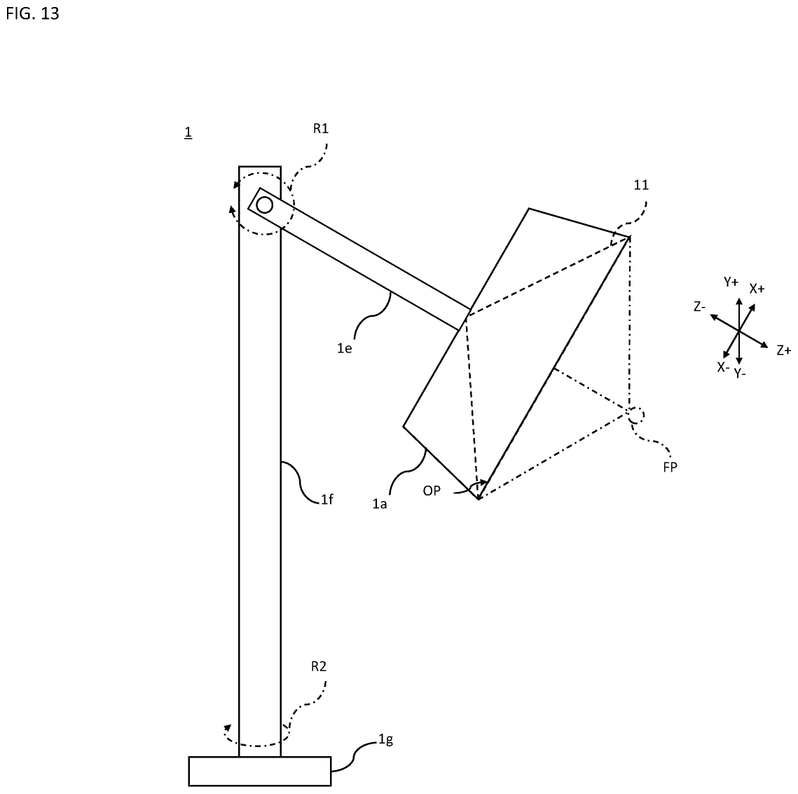

A third example of the external shape of the hair care apparatus 1 is described below. FIG. 13 shows a third example of the external shape of this hair care apparatus according to the present embodiment.

As shown in FIG. 13, the hair care apparatus 1 has a base cover 1a, a first arm 1e, a second arm 1f, and a pedestal 1g.

The base cover 1a has an opening OP. The base cover 1a is substantially trapezoidal in shape. Base cover 1a covers base 11. The base 11 is positioned where ultrasonic waves are radiated toward the aperture OP (i.e., in the Z+ direction).

The first arm 1e is connected to the base cover 1a, the second arm 1f, and the The first arm 1e is connected to the base cover 1a and the second arm 1f. Arm 1e extends in the Z direction.

The second arm if is connected to the first arm 1e and the pedestal 1g.

The second arm if supports the first arm 1e in a rotatable manner along the rotary axis R1. R1.

The pedestal 1g supports the second arm if rotatably along the rotation axis R2.

According to the hair care apparatus 1 shown in FIG. 13, the user of the hair care apparatus 1 uses the hair care apparatus 1 while placing it on a desk (i.e., without holding it in the hand). In this case, the user can use the hair care apparatus 1 while performing hair care by his hands (e.g., while applying a hair growth or hair regrowth agent or massaging).

### (4-4) Fourth Example of External Shape of Hair Care Apparatus

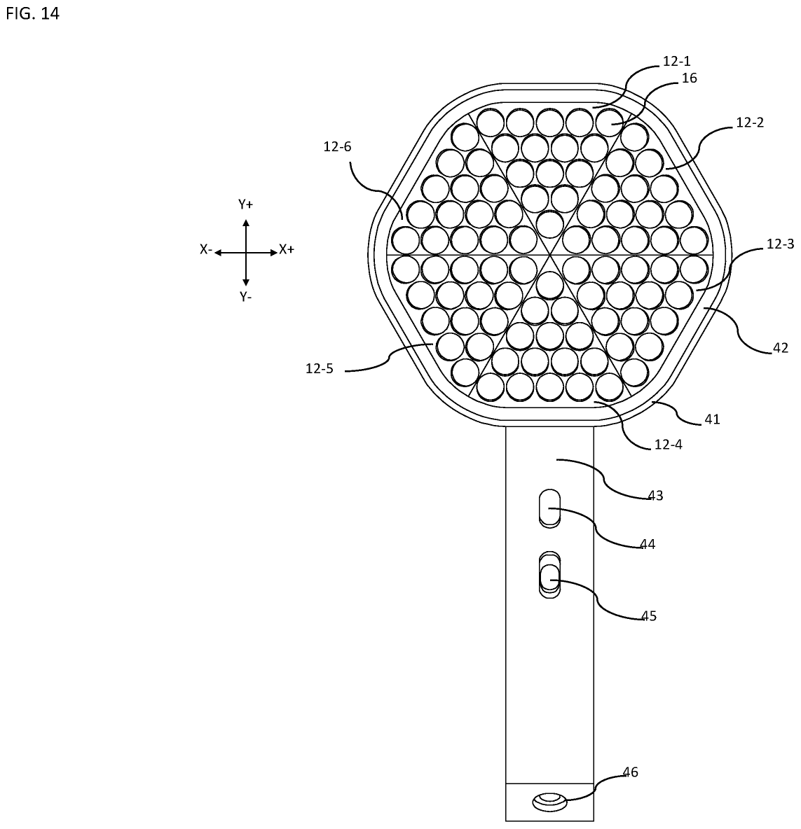

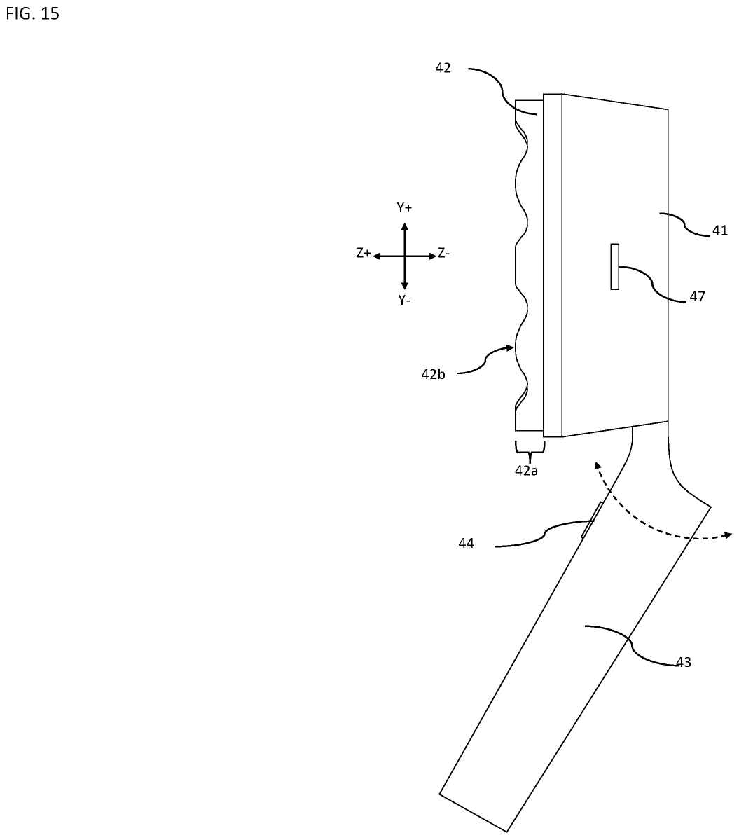

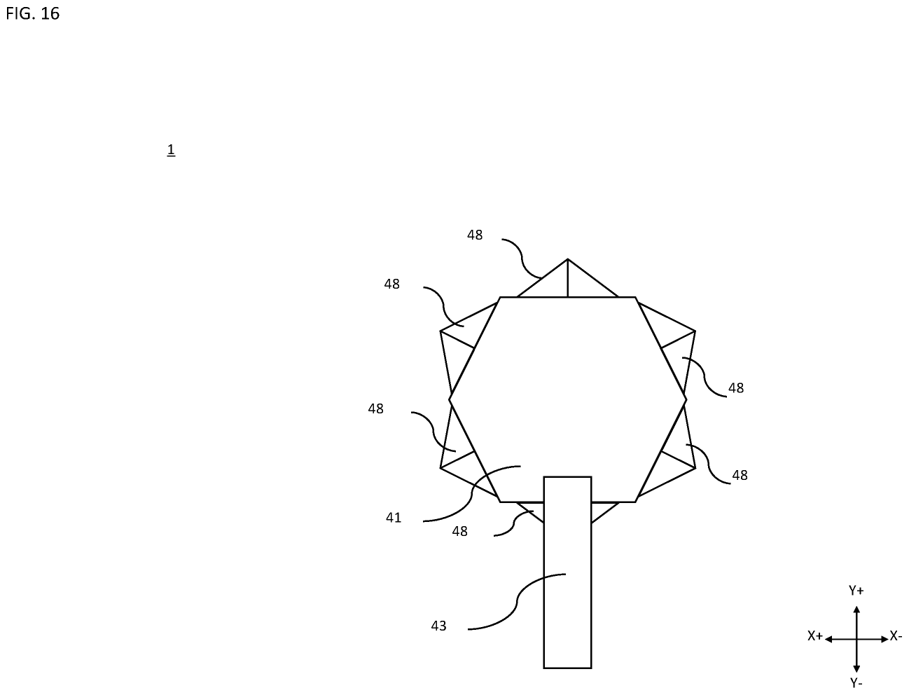

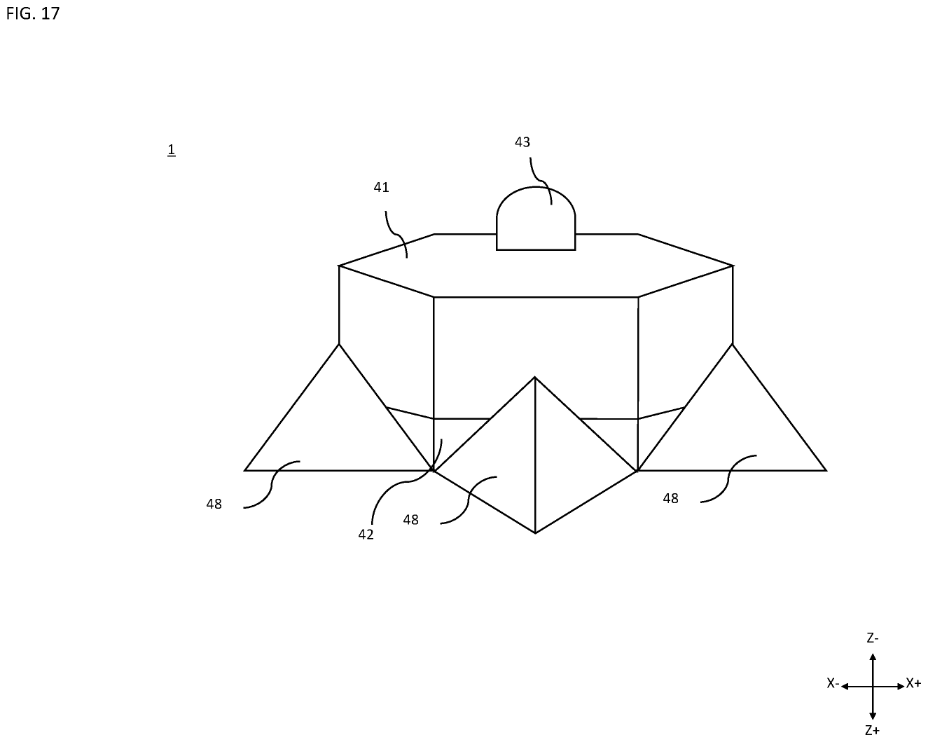

A fourth example of the external shape of the hair care apparatus 1 is described below. FIG. 14 is a front view of a fourth example of the external shape of the hair care apparatus according to the present embodiment. FIG. 15 is a side view of a fourth example of the external shape of the hair care apparatus according to the present embodiment. FIG. 16 shows an example of a head attachment. FIG. 17 shows an example of a head attachment.

As shown in FIGS. 14 and 15, the hair care apparatus 1 has six transducer supports 12-1 to 12-6, a head 41, a hood 42, a grip 43, a power button 44, a mode switch 45, a jack 46, and a lamp 47.

In this example, the ultrasonic radiation apparatus 10 has six transducer supports 12-1 to 1 2-6. The main face 12S of each transducer support 12 is triangular (e.g., equilateral or isosceles triangles). The triangle corresponding to the mains face 12S of the transducer support 12 shares one vertex with the triangles corresponding to the main faces 12S of all other transducer supports 12. The side (base) opposite the above vertex of the triangle corresponding to the main face 12S is arranged to form a hexagon (e.g., a regular hexagon).

Head 41 supports six transducer supports 12-1 to 12-6. The head 41 is similar in shape to a hexagonal cylinder with the bottom corresponding to an aperture and a hollow interior. The transducer support 12 is attached to the head 41. The transducer support 12 is housed inside the head 41 so that ultrasound can be radiated through the above aperture.

The hood 42 is attached to the head 41 so that it surrounds the aperture of the head 41, i.e., the six transducer supports 12-1 to 12-6, when the hair care apparatus 1 is viewed from the front. Hood 42 has an annular wall 42a protruding in the radiation direction (Z+ direction). The surface of the annular wall 42a through the plurality of tips 42b that protrude the most in the radiation direction is defined as the tip surface. The shape of the hood 42 is defined so that the common target point is included in the tip surface of the annular wall 42a. This allows the user to position the hair care apparatus 1 so that the plurality of tips 42b of the annular wall 42a are pressed the area around the affected area thereby aligning the target point with the affected area. In other words, the hood 42 serves as a guide to align the target point with the affected area.

In the example of FIGS. 14 and 15, the annular wall 42a is configured so that its radial dimension periodically increases or decreases according to its position on a plane orthogonal to the radiation direction (XY plane). This allows the area of the tip 42b that contacts the user's head when using the hair care apparatus 1 to be smaller. In other words, the portion of the hood 42 that is in contact with the human body can be reduced, which is hygienic, and the increased pressure on the head enhances the massage effect.

Hood 42 may be configured to be removable from head 41. This allows the hood 42 to be easily replaced and the hair care apparatus 1 to be used with the hood 42 removed. As an example, hood 42 is attached to head 41 by a magnet built into head 41 or hood 42.

The user can either remove hood 42 from head 41 or attach hood 42 to head 4 1, or with the hood 42 attached to the head 4 1, the user can attach a different head attachment to the head 41 or hood 42 than the hood 42. The user can attach a different head attachment to the head 41 or hood 42. As an example, the head attachment can be attached to the head 41, hood 42, or to the head 4 by means of a magnet built into said head attachment. 1 or hood 42.

An example of a head attachment is illustrated in FIGS. 16 and 17. Head attachment 48, shown in FIGS. 16 and 17, is mounted on the outer surface of the head 41 and hood 42. Head attachment 48 has a shape similar to a snow plow. The user moves the hair care apparatus 1 near the affected area while keeping the plurality of tips 42b of the annular wall 42a of the hood 42 in contact with the head. As a result, even if there is long hair around the affected area, the head attachment 48 pushes the hair away, allowing the ultrasonic waves radiated by the ultrasonic wave radiation apparatus 10 to reach the affected area without being blocked by the hair.

In the examples in FIGS. 16 and 17, the number of head attachments 48 corresponds to the number of transducer supports 12, but they may be different.

Grip 43 supports head 41. Grip 43 is configured to be graspable by the user. The grip 43 is configured to extend in a direction closer to the radiation direction (i.e., in the direction between the Z+ and Y− directions) than in a direction orthogonal to the radiation direction (e.g., in the Y− direction). direction (i.e., the direction between the Z+ and Y− directions). This allows the user to suppress the arm movements required to bring the ultrasonic radiation apparatus 10 housed in the head 41 against his or her own head.

Grip 43 is configured to rotate around the X-axis by means of a movable part not shown. This allows the user to adjust the angle of the grip 43 according to the position of the affected area to reduce arm strain.

The power button 44 is a physical button for turning the power of the hair care apparatus 1 on and off. The power button 44 is located on the grip 43. This allows the user to turn on/off the hair care apparatus 1 while holding the grip 43 with one hand.

The mode switch 45 is a physical switch for switching the operating mode of the hair care apparatus 1. The mode switch 45 is located on the grip 43. This allows the user to switch the operation mode of the hair care apparatus 1 while holding the grip 43 with one hand.

Jack 46 is a terminal for charging the battery (not shown) provided by the hair care apparatus 1. Jack 46 is provided at the end of grip 43 opposite head 41.

Lamp 47 is, for example, a Light Emitting Diode (LED). The lamp 47 is controlled by the controller 30 to light up according to the operating state of the hair care apparatus 1 (e.g., normal state, low battery state, etc.).

According to the hair care apparatus 1 shown in FIGS. 14 and 15, the tip 42b of the annular wall 42a of the hood 42 constitutes only a part of the aforementioned tip surface, so that the contact area between the hood 42 and the scalp is kept to the minimum necessary. Therefore, according to the hair care apparatus 1, the user grasps the grip 43 with one hand and operate the hair care apparatus 1 with the tips 42b of the annular wall 42a of the hood 42 pressing against the area around the affected area. This allows to the ultrasonic waves radiated from the ultrasonic wave radiation apparatus 10 to be focused on the affected area without having to pay detailed attention to the position or orientation of the hair care apparatus 1, while minimizing adverse effects on hygiene.

### (4-5) Fifth Example of External Shape of Hair Care Apparatus

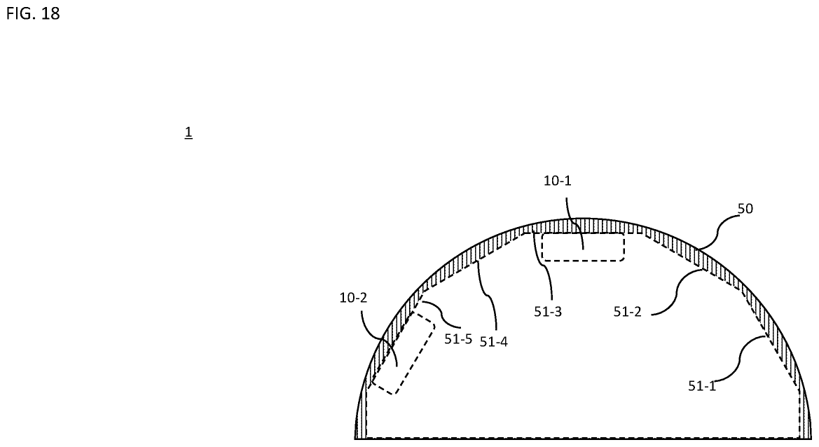

The following is a fifth example of the external shape of the hair care apparatus 1. FIG. 18 shows a fifth example of the external shape of this hair care apparatus.

As shown in FIG. 18, the hair care apparatus 1 has a housing 50.

The housing 50 can be worn on the human head. The housing 50 has a hollow hemispherical shape similar to a hat, for example. The housing 50 has a plurality of device attachment sections 51-1 through 51-5 at different positions on the surface opposite the head when worn on the human head.

The ultrasonic radiation apparatus 10 can be attached to and detached from the device attachment section 51. As an example, the ultrasonic radiation apparatus 10 is secured to the device attachment section by a magnet built into the ultrasonic radiation apparatus 10 or the device attachment section 51.

According to the hair care apparatus 1 shown in FIG. 18, the user can perform hair care while wearing the hair care apparatus 1 on the head. This means that the user can use both hands freely while performing hair care. By attaching multiple ultrasonic radiation apparatuses 10 to multiple device attachment sections 51, hair care for multiple affected areas can be performed simultaneously and hair care can be completed in a short time.

The housing 50 may have a shape similar to headphones rather than a hat. In this case, the housing 50 may have a band-shaped headband with a plurality of device attachment sections 51 and pads that secure the ends of the headband to the user's head (e.g., both ears).

### (5) Summary

As explained above, the ultrasonic radiation apparatus 10 of the present embodiment has a plurality of transducer supports 12 and a plurality of ultrasonic transducers 16 mounted on each transducer support 12. The transducer support 12 has a main face 12S and a plurality of mounts 14 on the main surface 12S, the plurality of ultrasonic transducers 16 being able to be mounted on the plurality of mounts 14. The transducer support 12 is arranged such that the normal extending from the main face 12S of the transducer support 12 toward the direction of ultrasonic waves intersects the normal extending from the main face 12S of each of the other transducer supports 12 to the direction of ultrasonic waves at a common target point. This allows the ultrasonic waves radiated by the ultrasonic transducers 16 mounted on each transducer support 12 to be focused on the aforementioned target point. In other words, by aligning the treatment area of the subject with the target point, acoustic radiation pressure can be applied to the treatment area in a non-contact manner. Therefore, the application of the ultrasonic radiation apparatus 10 in the present embodiment to a hair care apparatus can reduce adverse effects on hygiene in hair care.

Each of the plurality of ultrasonic transducers 16 may be mounted at offset position such that the greater the distance from the reference point on the main surface 12S of the transducer support 12 to the mount 14 corresponding to the ultrasonic transducer 16, the greater its distance from the main surface 12S with respect to a direction orthogonal to the main surface 12S. This reduces the variation in the distance from a group of ultrasonic transducers 16 mounted on each transducer support 12 to the aforementioned target point and focuses the ultrasonic waves on the target point.

The attachment positions of the plurality of ultrasonic transducers 16 on the transducer support 12 may be determined so that the distances from a point at a predetermined distance away from the main face 12S may be determined are substantially equal to each other. This allows the distance from a point a predetermined distance away from the main face 12S to each ultrasonic transducer 16 to be homogenized for each transducer support 12 to align the phase of the ultrasonic waves at that point.

The transducer support 12 may have at least one attachment 13 mounted on the main face 12S, the attachment 13 forming a stepped staircase-like region with respect to a direction perpendicular to the main face 12S. At least one of the plurality of ultrasonic transducers 16 may be attached to the corresponding mount 14 via the attachment 13. This allows the distance from the main face 12S to each ultrasonic transducer 16 to be easily adjusted.

One of the plurality of ultrasonic transducers 16 mounted on the transducer support 12 may radiate ultrasonic waves traveling along the aforementioned normal at the main face 12S. This allows the ultrasonic wave radiated from this ultrasonic transducer 16 to reach the aforementioned target point in the shortest path, thereby increasing energy efficiency.

Each transducer support 12 may be arranged so that the distance from the main face 12S of the transducer support 12 to the target point is substantially equal to each other. This allows the variation of the distance from the aforementioned target point to each ultrasonic transducer 16 to be suppressed over all transducer supports 12, and the ultrasonic waves can be focused more reliably on the target point.

The hair care apparatus 1 according to the present embodiment is equipped with an ultrasonic radiation apparatus 10 and a controller 30 that drives the plurality of ultrasonic transducers 16 simultaneously. This reduces the negative impact on hygiene in hair care.

The hair care apparatus 1 is preferably used after applying a hair growth or hair regrowth agent. According to the present embodiment, the acoustic radiation pressure by the focused ultrasound increases the penetration rate of the hair growth or hair regrowth agent into the scalp. As a result, the hair growth or hair regrowth effect can be further promoted.

### (6) Modification

Modifications of the present embodiment are described.

### (6-1) Modification 1

Modification 1 is described. Modification 1 is an example of a means for notifying that ultrasonic waves are being radiated.

### (6-1-1) Configuration of Hair Care Apparatus

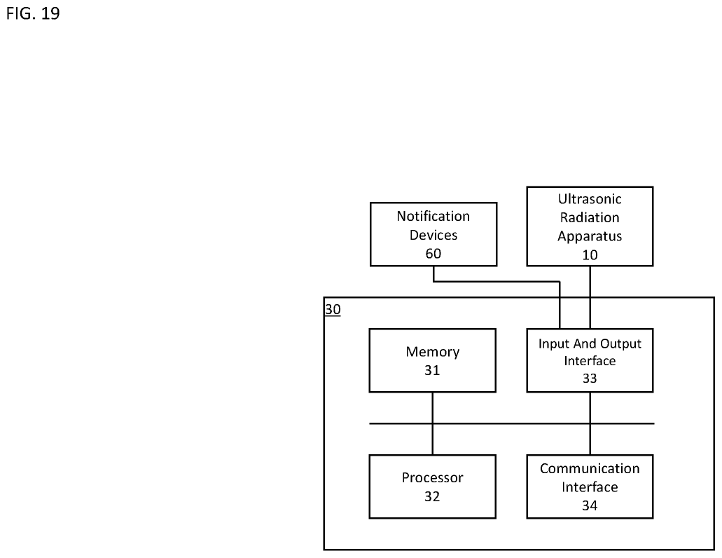

The configuration of the hair care apparatus in Modification 1 is described below. FIG. 19 shows a block diagram of the hair care apparatus of Modification 1.

As shown in FIG. 19, the notification device 60 is connected to the controller 30. The notification device 60 can include, for example, at least one of the following:

a light emission element

a speaker; and

a vibration device.

The notification device 60 operates according to control from the controller 30 while the ultrasonic radiation apparatus 10 is radiating ultrasound. Specifically, the notification device 60 informs the user that ultrasound is being radiated, for example, by generating at least one of light, sound, or vibration. When the notification device 60 is equipped with a light emission element, the notification device 60 is preferably arranged at a location visible to the user during use of the hair care apparatus 1 (e.g., at the bottom of the grip 43 of the hair care apparatus 1 in FIG. 14).

As explained above, the hair care apparatus 1 of Modification 1 is equipped with a notification device 60. This allows the user to intuitively understand the operating state of the hair care apparatus 1 (i.e., whether or not it is radiating ultrasound).

The lamp 47 of the hair care apparatus 1 in FIGS. 14 and 15 can be used as the light emission device.

Further modifications of the present modification are possible.

As a first example, the controller 30 may detect whether the target point (i.e., the focal point of the ultrasound radiated by the ultrasonic radiation apparatus 10) coincides with the affected area of the user (e.g., scalp) and change the operation of the notification device 60 accordingly. As an example, the notification device 60 differs in the content (e.g., pattern) of the light, sound, or vibration it generates when the target point coincides with the user's affected area and when it does not.

As a second example, instead of or in addition to the notification device 60, the hair care apparatus 1 may be equipped with a lightweight part that is positioned in a location that overlaps the path of the ultrasonic waves radiated the ultrasonic wave radiation apparatus 10. Such parts vibrate by ultrasonic waves and thus produce the same effect of notifying ultrasonic radiation to the user as the notification device 60.

### (6-2) Modification 2

Modification 2 is described. Modification 2 is an example of a hair dryer configured as a hair care apparatus 1.

### (6-2-1) Configuration of Hair Care Apparatus

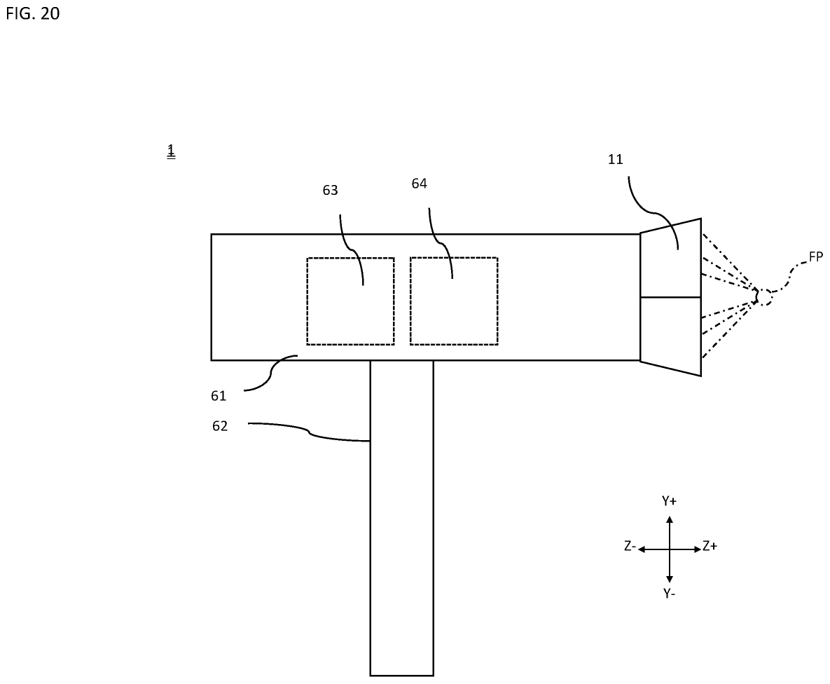

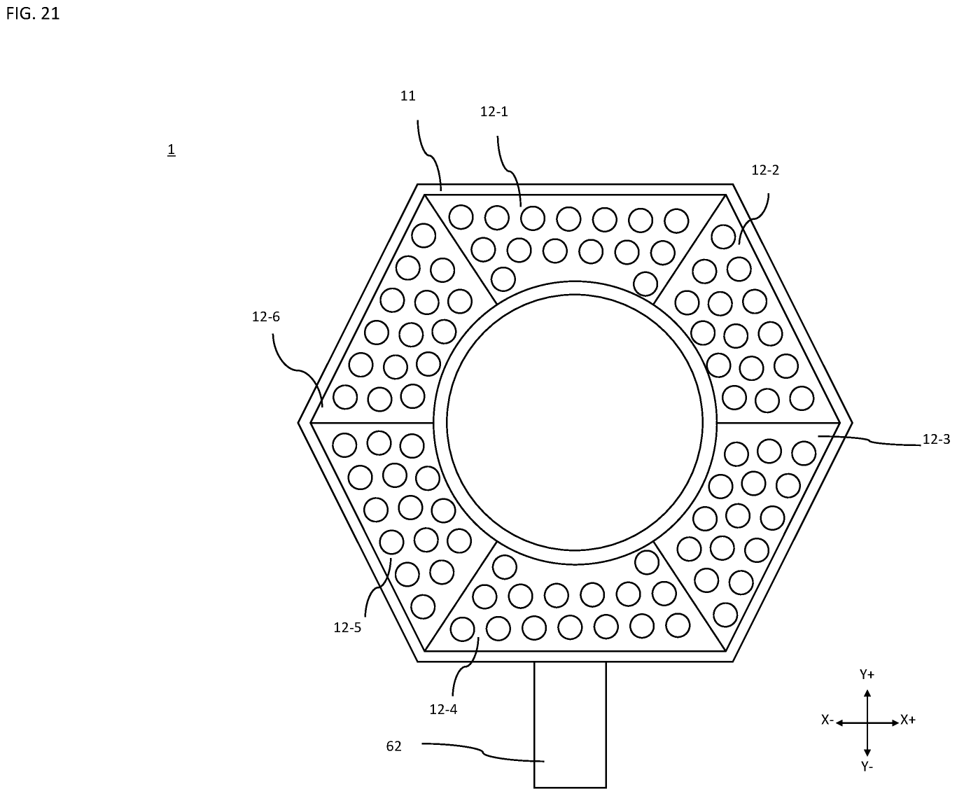

The configuration of the hair care apparatus in Modification 2 is described below. FIG. 20 shows a side view of the hair care apparatus in Modification 2. FIG. 21 is a front view of the hair care apparatus in Modification 2.

As shown in FIGS. 20 and 21, the hair care apparatus 1 has a head 61, a grip 62, an air blower 63, and a heater 64.

The head 61 has a shape similar to a cylinder where the bottom of the radial (Z+) side corresponds to the opening end and the bottom of the Z− side corresponds to the closed end.

The head 61 is mounted with the ultrasonic radiation apparatus 10 on the outer surface of the end in the Z+ direction. The transducer supports 12-1 to 12-6 are each arranged so that the normals of the main faces of the transducer supports 12 intersect at a common target point (focal point) FP. The head 61 houses an air blower 63 and heater 64 in its interior space.

Grip 62 extends in a substantially orthogonal (Y−) direction to the radial (Z+) direction. Grip 62 may have a power button, mode switch, or other operation parts not shown.

The blower 63 includes, for example, a fan and a motor. The blower 63 generates wind in the radiation direction (Z+ direction) in response to control from the controller 30.

Heater 64 is provided on the radial (Z+) direction side compared to blower 63. Heater 64 generates heat in response to control from controller 30.

The controller 30 turns on the blower 63 and heater 64 to and allows warm air to flow in the radiation direction (Z+ direction) from the opening of the head 61 (operation in warm air mode). The controller 30 turns on the blower 63 and turns off the heater 64 to allows air with a lower temperature than the warm air can flow from the opening of head 61 in the radiation direction (operation in cool air mode).

According to the hair care apparatus 1 in Modification 2, the user can select to perform hair care, hair drying, or both as needed.

### (6-3) Modification 3

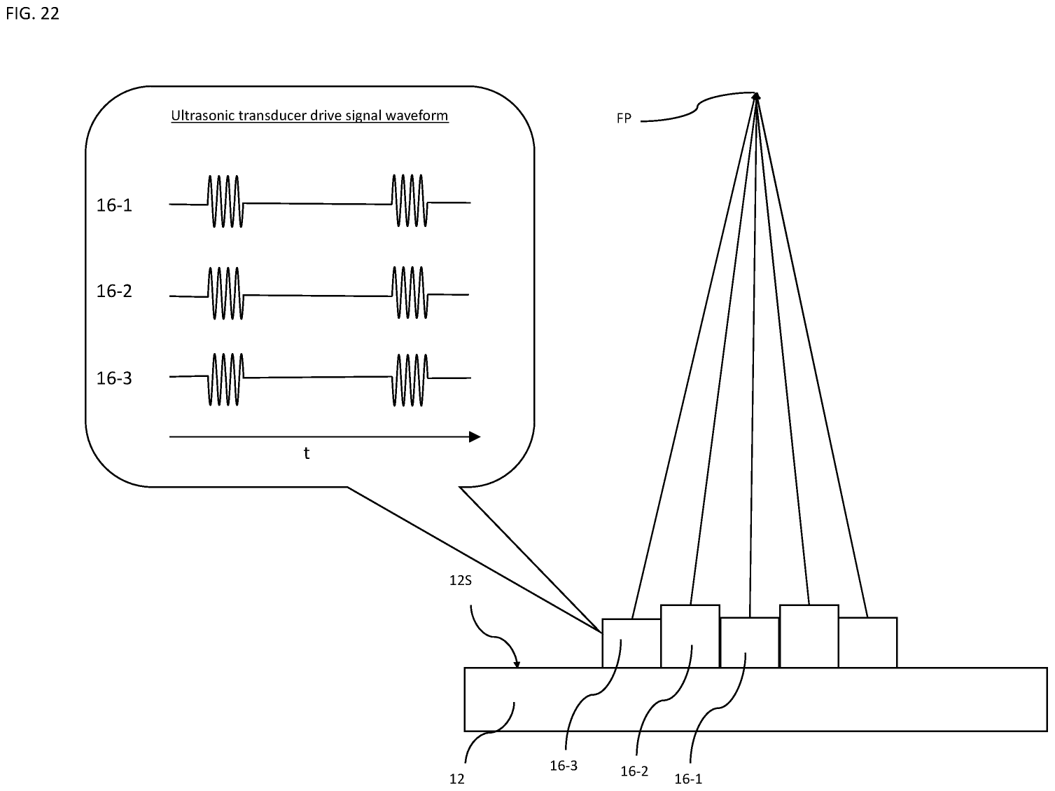

Modification 3 is described. Modification 3 is an example where some ultrasonic transducers are driven in opposite phase with other ultrasonic transducers. FIG. 22 illustrates an aspect of Modification 3.

In FIG. 8 of the present embodiment, a plurality of ultrasonic transducers 16 are mounted on the main face 12S of the transducer support 12 so that the distances from the point FP, which is a predetermined distance away from the main face 12S, to the ultrasonic transducers 16 are substantially equal. However, by driving some of the ultrasonic transducers 16 in opposite phase with the other ultrasonic transducers 16, the distance from the some of the ultrasonic transducers 16 (from the mount position) to the point FP can be different from the distance from the other ultrasonic transducers 16 to the point FP.

Specifically, as shown in FIG. 22, suppose that the distance from point FP to ultrasonic transducer 16-3 is configured to be about half a wavelength longer than the distance from point FP to ultrasonic transducers 16-1 and 16-2, and ultrasonic transducer 16-3 is driven in opposite phase with ultrasonic transducers 16-1 and 16-2 at the substantially same time. In this case, as in the example of FIG. 8, the ultrasonic waves radiated from each ultrasonic transducer 16 are in phase at the point FP and strengthen each other, and furthermore the ultrasonic transducer 16-3 can be made lower in height than in the example of FIG. 8.

According to an example described in the present embodiment, each of the plurality of ultrasonic transducers 16 is mounted at an offset position such that the greater the distance from the reference point on the main face 12S (e.g., the position of the mount 14-1) to the mount 14 corresponding to the ultrasonic transducer 16, the greater the distance from the main surface 12S with respect to a direction orthogonal to the main face 12. In the present modification 3, for example, the ultrasonic transducer 16 corresponding to the mount 14 with the greatest distance from the reference point is driven in opposite phase to the ultrasonic transducer 16 corresponding to the mount 14 with the smallest distance from the reference point, thereby the maximum distance between the ultrasonic transducers 16 and the main face 12S can be reduced.

As in Modification 3, some ultrasonic transducers 16 that are mounted on the main face 12S of the transducer support 12 are driven in opposite phase with the other ultrasonic transducers 16, thereby it is possible to converge ultrasonic waves radiated from each ultrasonic transducer 16 at the focal point by simple control (simultaneous driving) while suppressing the variation in the distance between the ultrasonic transducers 16 and the main face 12S.

### (6-4) Modification 4

Modification 4is described. Modification 4 is an example of a hair care apparatus 1 equipped with a dispense mechanism that dispenses hair care agents.

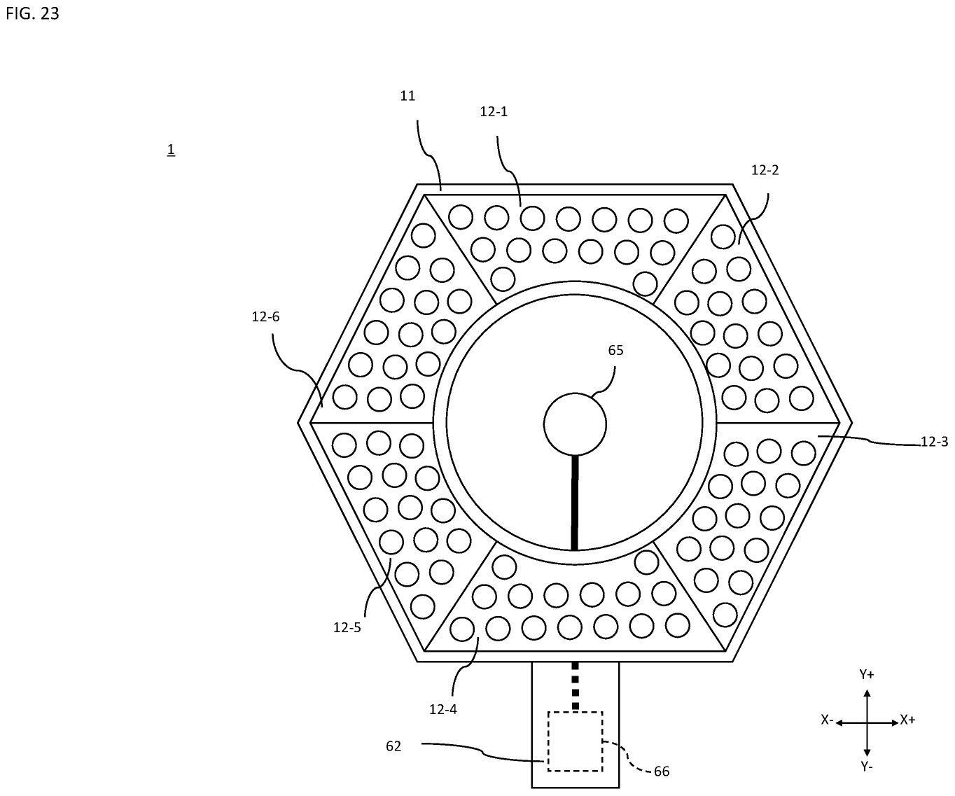

FIG. 23 shows a front view of the hair care apparatus in Modification 4.

As shown in FIG. 23, the hair care apparatus 1 of Modification 4 is equipped with a dispense mechanism that dispenses hair care agents. The dispense mechanism may have a nozzle 65 and a cartridge 66.

The cartridge 66 is removably attached to the hair care apparatus 1 (e.g., grip 62). Cartridge 66 is configured to contain hair care agents.

Hair care agents are substances (e.g., liquid substances) containing hair-growth or hair-regrowth ingredients. Hair care agents are, for example, minoxidil.

The nozzles 65 are located at the openings of the head 61 (i.e., in the area that does not overlap with the transducer supports 12-1 to 12-6) in the XY plane. Nozzle 65 is configured to be able to dispense hair care agents contained in cartridge 66 in the same direction as the ultrasonic radiation direction.

The hair care agent dispensed from nozzle 65 is applied to at least one of the user's scalp and hair at least one of before, during, and after the treatment.

In response to user operation (e.g., pulling the trigger) or in response to control from controller 30, the hair care agent contained in cartridge 66 is supplied to nozzle 65 and is dispensed from the tip of the nozzle 65.

Nozzle 65 is not limited to the position shown in FIG. 23 and may be integrated with, for example, head 41, hood 42 or head attachment 48, or may be attached to them.

According to Modification 4, in addition to non-contact vibratory pressure stimulation, a hair care agent is applied to the scalp, thereby the non-contact vibratory pressure stimulation is provided to the scalp where hair care agents are applied. As a result, the effect of the hair care agent is added to the effect of the non-contact vibratory pressure stimulation alone, thus enhancing the effect of hair growth.

### (6-5) Modification 5

Modification 5 is described. Modification 5 is an example of presenting to the user that a non-contact vibration pressure stimulus is being applied.

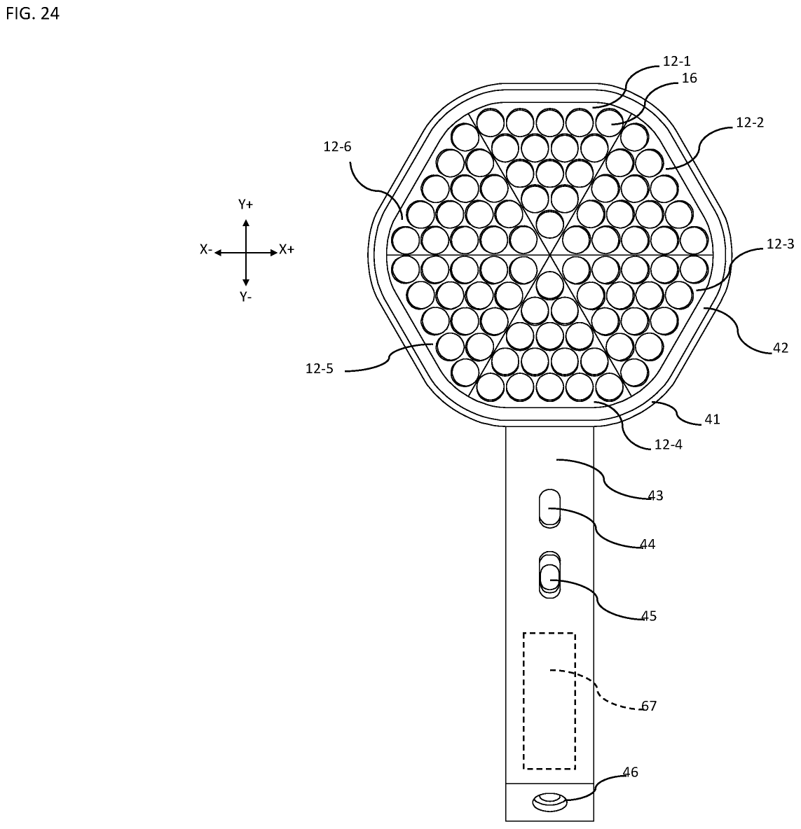

FIG. 24 shows a front view of the hair care apparatus in Modification 5.

As shown in FIG. 24, in the grip 43 of the hair care apparatus 1 of Modification 5, the vibration motor 67 is placed.

The vibration motor 67 vibrates when ultrasonic waves are radiated from the ultrasonic transducer 16.

The first example of the vibration pattern of the vibration motor 67 is described below. In the first example of the vibration pattern, the vibration motor 67 continues to vibrate while ultrasound is being radiated.

The second example of the vibration pattern of the vibration motor 67 is described below. In the second example of the vibration pattern, the vibration motor 67 vibrates in a vibration pattern corresponding to the elapsed time each time a predetermined time elapses from the start of ultrasonic radiation. For example, the vibration motor 67 will vibrate for 2 seconds every minute until 4 minutes have elapsed from the start of the radiation, and will vibrate for 10 seconds after 5 minutes have elapsed from the start of the radiation.

According to Modification 5, the vibration of the vibration motor 67 can tactilely present to the user that the treatment by the hair care apparatus 1 is being applied. In particular, the hair care apparatus 1 radiates ultrasonic waves (i.e., sound waves in a bandwidth inaudible to the user), so the presentation by the vibration motor 67 is particularly effective.

### (6-6) Modification 6

Modification 6 is described. Modification 6 is a modification on the configuration of the ultrasonic radiation apparatus.

### (6-6-1) Configuration of Ultrasonic Radiation Apparatus

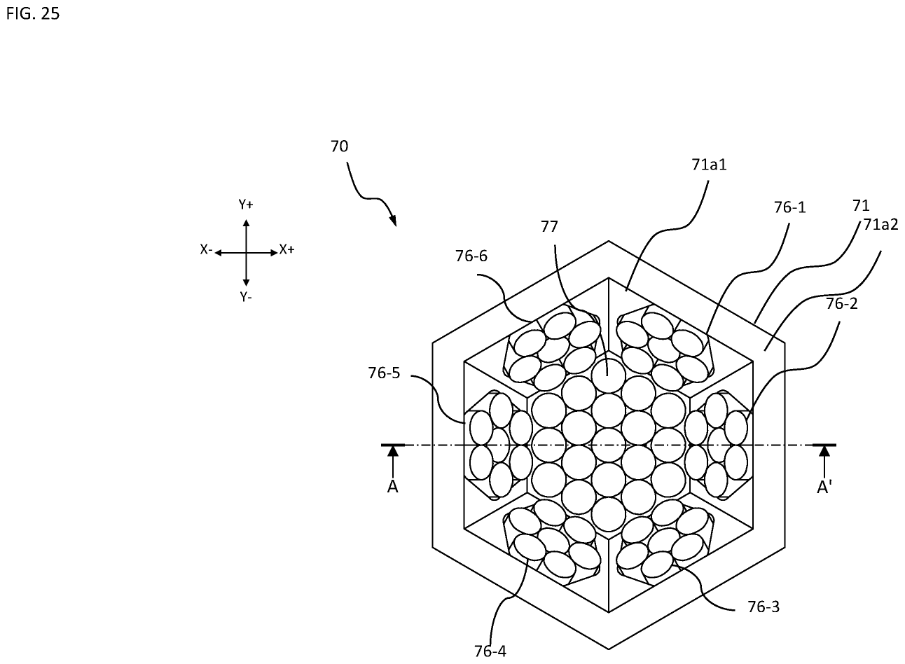

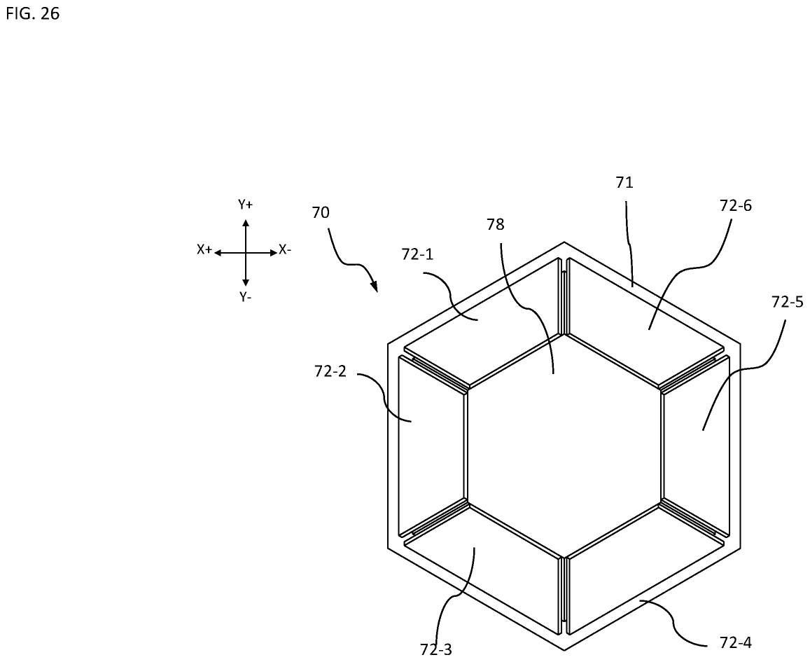

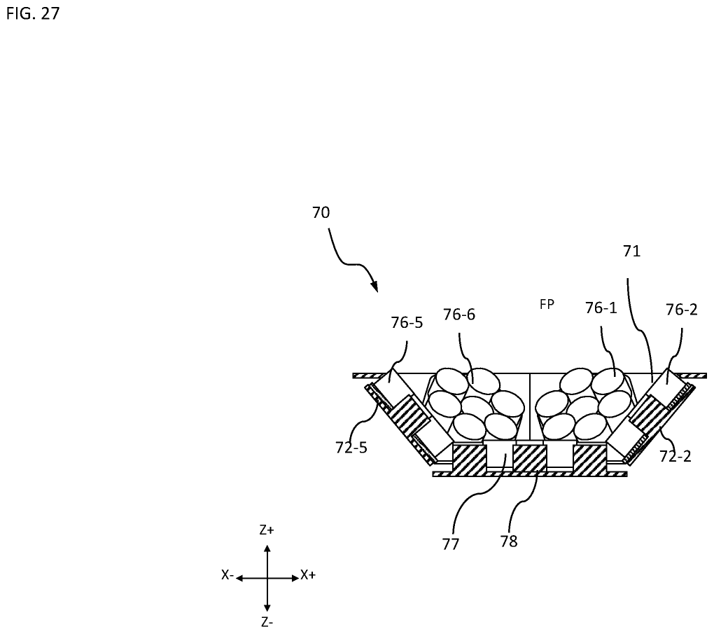

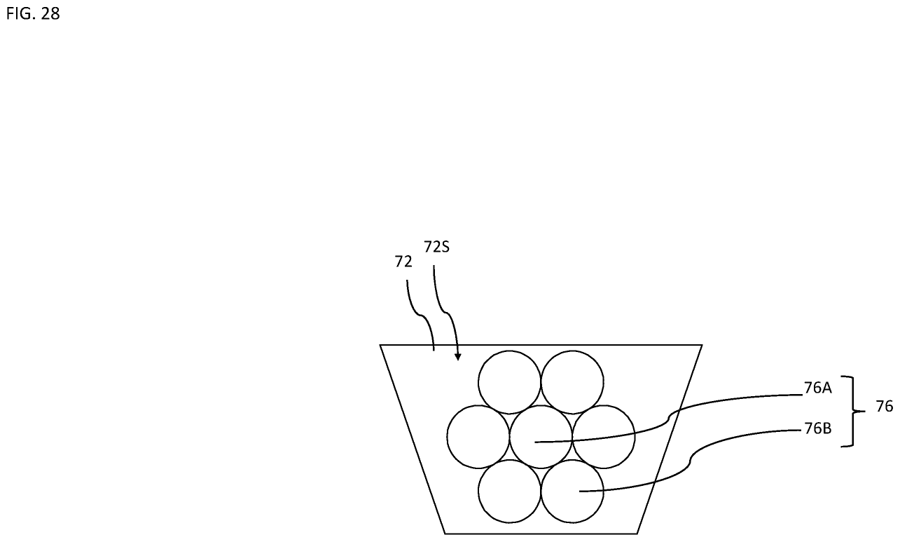

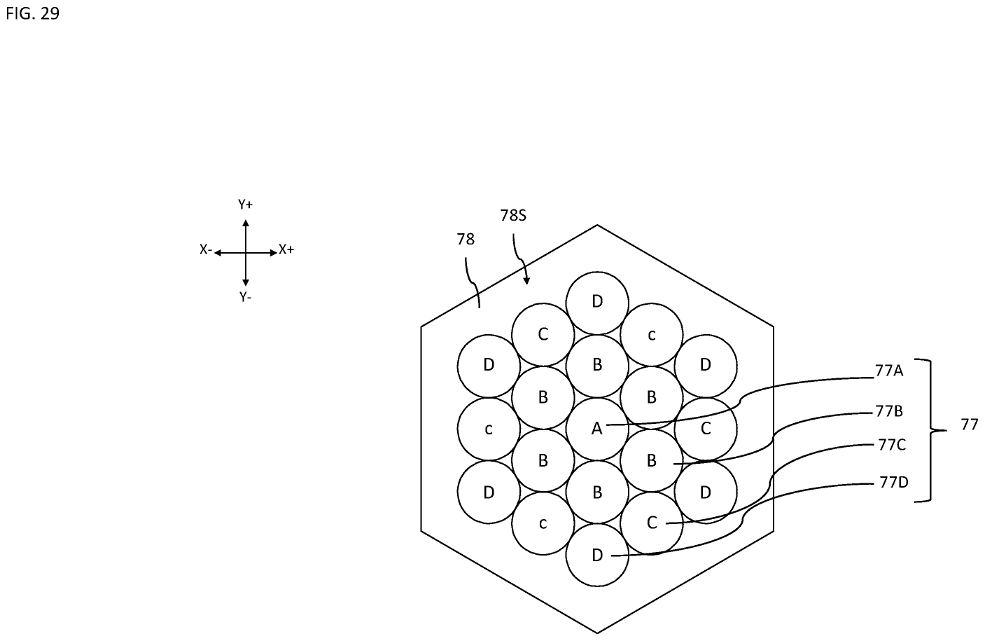

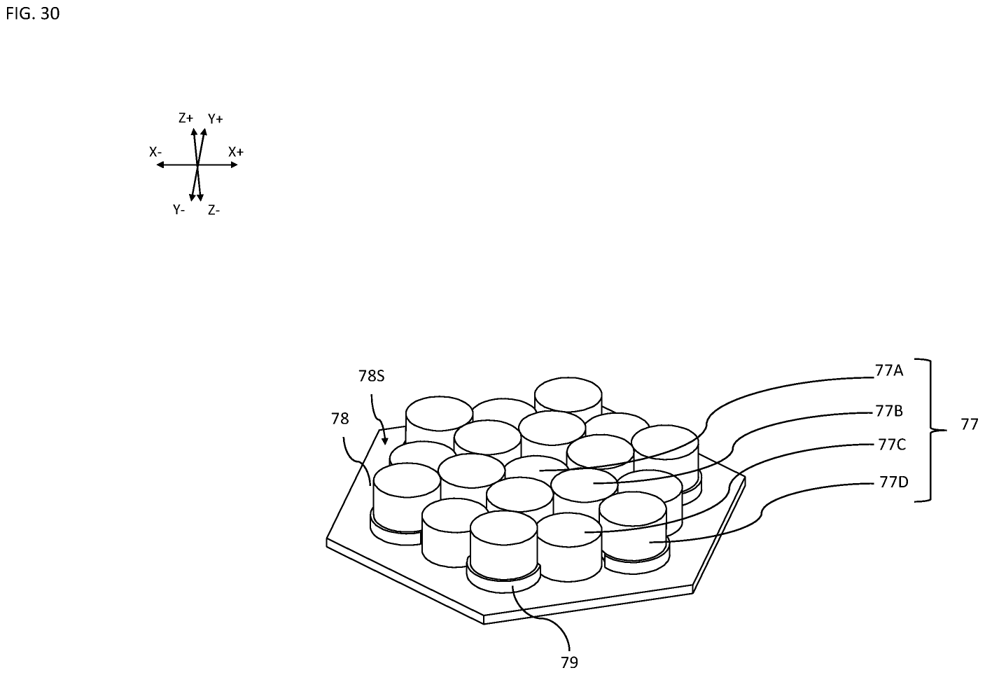

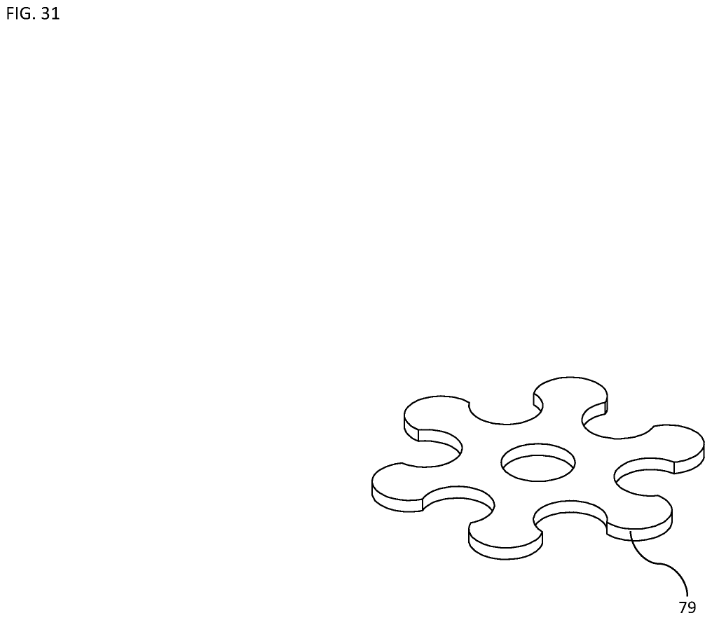

The configuration of the ultrasonic radiation apparatus in Modification 6 is described below. FIG. 25 shows the appearance of the ultrasonic radiation apparatus in Modification 6. FIG. 26 shows the appearance of the ultrasonic radiation apparatus in Modification 6. FIG. 27 illustrates the target points of the side and bottom supports provided by the ultrasonic radiation apparatus in Modification 6. FIG. 28 shows a plurality of ultrasonic transducers attached to the side supports of Modification 6. FIG. 29 shows the multiple ultrasonic transducers attached to the bottom support in Modification 6. FIG. 30 shows the multiple ultrasonic transducers attached to the bottom support in Modification 6. FIG. 31 shows the appearance of the attachment in Modification 6.

As shown in FIGS. 25 and 26, the ultrasonic radiation apparatus 70 comprises a base 71 and six side supports 72-1, 72-2, 72 72-3, 72-4, 7 2-5, 72-6, and a bottom support 78. As shown in FIG. 28, the ultrasonic radiation apparatus 70 has a plurality of ultrasonic transducers 76 for each side support 72. Although not shown in the figure, the ultrasonic radiation apparatus 70 has an attachment (spacer), a plurality of mounts, and a connector for each side support 72. As shown in FIGS. 29 and 30, the ultrasonic radiation apparatus 70 has a bottom support 78 on which a plurality of ultrasonic transducers 77 and attachments 79 are provided. Although not shown in the figure, the ultrasonic radiation apparatus 70 has a number of mounts and a connector on the bottom support 78.

The base 71 includes a first region 71a1 and a second region 71a2. The second region 71a2 is, for example, a substantially planar face 71a2. In the first region 71a1, a plurality of polygonal faces that differ from each other in at least one of their positions and orientations are combined. The combination of the multiple polygonal faces constitutes a three-dimensional shape that is convex in the Z− direction. A plurality of polygonal faces are combined so that the normal extending from each face to the direction of ultrasound travel intersects one of the normals extending from the other faces to the direction of ultrasound travel at a common point of intersection. As an example, as shown in FIGS. 25 and 26, in the first region 71a1, a substantially regular hexagonal face and six trapezoidal faces that share a base (the shorter base) with each side of the substantially regular hexagonal face are combined. The combination of a substantially regular hexagonal face and six trapezoidal faces is an umbrella-like three-dimensional shape convex in the Z− direction. The second region 71a2 surrounds the first region 71a1.

The base 71 has means (e.g., screw holes) for attaching each of the side supports 72 and the bottom support 78 to on each of the surfaces comprising the first region 71a1. The base 71, with the side supports 72 and the bottom support 78 attached, is configured to support the side supports 72 and bottom support 78.

The base 71 has means (e.g., screw holes) for attachment to an object not shown (e.g., the housing of the hair care apparatus 1), for example, in the second region 71a2. With the base 71 attached to an object, the base 71 can maintain its position and orientation (e.g., the angle of rotation around the X, Y, and Z-axis) with respect to that object can be maintained (i.e., stationary).

As shown in FIG. 28, the side support 72 has a substantially planar main face 72S, and the side support 72 is attached to each face (side) of the first region 71a1 of the base 71 so that the main face 72S faces the direction of ultrasonic waves. The side support 72 is arranged such that its main face 72S is non-parallel to the main faces 72S of the other side supports 72 (in other words the plane including the main face 72S of the side support 72 does not coincide with the planes including the main faces 72S of the other side supports 72). The side support 72 is arranged so that a normal extending from the main face 72S of the side support 72 to the direction of ultrasonic wave radiated by the ultrasonic transducer 76 does not intersect the main face 78S of the bottom support 78. The side support 72 has means (e.g., screw holes) for attaching the side support 72 to the base 71. With the side supports 72 attached to the base 71, the side supports 72 can maintain their position and orientation (i.e., remain stationary) relative to the base 71. Specifically, the side support 72 is mounted on the base 71 so that one of the normals extending from the main face 72S to the direction of ultrasonic wave travel intersects at a common target point with one of the normals extending from the main face 72S of another side support 72 to the direction of ultrasonic wave travel and one of the normals extending from the main face (described below) of the bottom support 78. The target point may coincide with or differ from the aforementioned intersection point. If the main face 72S of the side support 72 is parallel to the face in the first region 71a1 of the base 71 to which the side support 72 is attached, the aforementioned intersection point coincides with the target point. Furthermore, the side supports 72 may be mounted on the base 71 such that the distances from the main faces 72S of the side supports 72 to the aforementioned target point are substantially coincident with each other.

The main face 72S is identical or similar in shape to the (side) face constituting the first region 71a1 of the base 71. As an example, the main face 72S is trapezoidal.

Although not shown in the figure, the side support 72 has a plurality of mounts on the main face 72S. The mount is configured to allow mounting of the ultrasonic transducer 76. With the ultrasonic transducer 76 attached to the mount the side support 72 is configured to support the ultrasonic transducer 76 and to provide electrical connection to the connector 15.

More precisely, the plurality of ultrasonic transducers 76 are classified into a plurality of groups based on the distance from the reference point to the attachment position of the ultrasonic transducer 76 (here, the distance in the same plane as the main face 72S). Here, the reference point is, for example, the point where a perpendicular line descending from a target point determined by attaching the side support 72 to the base 71 to the main face 72S intersects the main face 72S.

The ultrasonic transducers 76 belonging to each group are configured to be different from the ultrasonic transducers 76 belonging to the other groups in terms of at least one of the distance from the main face 72S (i.e., the attachment position with respect to the direction orthogonal to the main face 72S), or whether or not the drive signal is inverted input.

The following description describes an example in which the ultrasonic transducer 76A corresponding to the attachment position whose distance from the reference point is Da1 is classified into group A, and the ultrasonic transducer 76B corresponding to the attachment position whose distance from the reference point is Db1 (Db1>Da1) is classified into group B. In this example, the ultrasonic transducers 76 belonging to group A are attached at a different distance from the main face 72S with respect to the direction orthogonal to the main face 72S than the ultrasonic transducers 76 belonging to group B.

The attachment position with respect to the direction orthogonal to the main face 72S can be adjusted by an attachment not shown. An attachment is mounted on the main face 72S of the side support 72. The attachment is configured to adjust the distance between the ultrasonic transducer 76 belonging to group B and the main face 72S. Specifically, the attachment has an substantial uniform thickness and provides a substantially planar area above the main face 72S that is spaced apart by a distance corresponding to the thickness with respect to a direction perpendicular to the main face 72S.

The ultrasonic transducers belonging 76 to group A are directly attached to the mount. On the other hand, the ultrasonic transducers 76B belonging to group B are attached to the mount via attachments. Therefore, the distance from each ultrasonic transducer 76 to the main face 72S is not uniform. In other words, each ultrasonic transducer 76 is attached to the side support 72 at a different distance from the main face 72 than at least one of the other ultrasonic transducers 76. As an example, each of the plurality of ultrasonic transducers 76 is arranged at an offset position such that the greater the distance from a reference point on the main face 72S to the mount corresponding to the ultrasonic transducer 76, the greater the distance from the main face 72S with respect to a direction perpendicular to the main face 72S. Specifically, the attachment position of each ultrasonic transducer 76 can be determined as a position on an substantially spherical surface centered at a point a certain distance away from the main face 72S (the point corresponds to the target point with the side support 72 attached to the base 71, and is hereinafter referred to as the “designed focal point of the ultrasonic transducers 76”). In other words, the attachment positions of the plurality of ultrasonic transducers 76 can be defined so that the distance from the designed focal point of the ultrasonic transducers 76 is substantially equal to each other.

Although it is not shown in the drawings, side support 72 supports the connector on the main face 72S. The connector is connected to an external device (e.g., controller 30) to enable electrical signals to be exchanged.

With the ultrasonic transducer 76 attached (directly or indirectly via an attachment) to the mount, the ultrasonic transducer 76 can maintain its position and orientation (i.e., remain stationary) against the side support 72. The ultrasonic transducer 76 is electrically connected to the connector while attached to the mount. The ultrasonic transducer 76 is configured to receive, via the connector, a drive signal transmitted by the controller 30 and radiate ultrasonic waves traveling along the first direction in accordance with the drive signal. The first direction is, for example, normal to the main face 72S of the side support 72. The ultrasonic waves radiated from each of the ultrasonic transducers 76 converge at the focal point FP, which corresponds to the aforementioned designed focal point, as shown in FIG. 27.

As shown in FIGS. 29 and 30, the bottom support 78 has a substantially planar main face 78S, and is attached to one side (bottom surface) of the first region 71a1 of the base 71 so that the main face 78S faces the direction in which the ultrasonic waves are traveling. The bottom support 78 has means (e.g., screw holes) for attaching the bottom support 78 to the base 71. The bottom support 78 is arranged such that the main face 78S of the bottom support 78 is non-parallel to the main face 72S of each side support 72 (That is, a plane including the main face 78S of the bottom support 78 does not coincide with a plane including the main face 72S of each of the side supports 72). With the bottom support 78 attached to the base 71, the bottom support 78 can maintain its position and orientation (i.e., remain stationary) with respect to the base 71. Specifically, the bottom support 78 is arranged on the base 71 such that one of the normals extending from the main face 78S to the direction of travel of the ultrasound intersects at a common target point with one of the normals extending from the main face 72S of each of the side supports 72 to the direction of travel of the ultrasound. The target point may coincide with or differ from the aforementioned intersection point. If the main face 78S of the bottom support 78 is in parallel to the surface of the first region 71a1 of base 71 to which the bottom support 78 is attached, the aforementioned intersection point coincides with the target point. Furthermore, the bottom support 78 may be mounted on the base 71 so that the distance from the main face 78S of the bottom support 78 to the aforementioned target point substantially equals to the distance from the main face 72S of each side support 72 to the target point.

In other words, the planes including the main faces of each support (bottom support 78 and each side support 72) are arranged so that they are non-parallel (i.e., they do not coincide) with each other.

The main face 78S is identical or similar in shape to the surface (bottom surface) constituting the first region 71a1 of the base 71. As an example, the main face 78S is a substantially regular hexagonal shape. The six side supports 72 are arranged such that the trapezoid corresponding to the main face 72S of each side supports 72 shares a first base (the shorter base) with a side of the hexagon corresponding to the main face 78S of the bottom support 78 and the second bases (longer bases) of the trapezoids corresponding to the main faces 72S of the six side supports 72 form a hexagon larger than the main face 78S of the bottom support 78.

Although not shown in the figure, the bottom support 78 has a plurality of mounts on the main face 78S. The mount is configured to allow mounting of the ultrasonic transducer 77. The bottom support 78 is configured to support the ultrasonic transducer 77 with the ultrasonic transducer 77 attached to the mount and to provide an electrical connection to the connector.

More precisely, a plurality of ultrasonic transducers 77 are classified into a plurality of groups based on the distance from the reference point to the attachment position of the ultrasonic transducer 77 (in this case, the distance in the same plane as the main face 78S). Here, the reference point is, for example, the point where a perpendicular line descending from a target point determined by attaching the bottom support 78 to the base 71 to the main face 78S S intersects the main face 78S.

The ultrasonic transducers 77 belonging to each group are configured to be different from the ultrasonic transducers 77 belonging to the other groups in terms of at least one of the distance from the main face 78S (i.e., the attachment position with respect to the direction orthogonal to the main face 78S), or whether or not the drive signal is inverted input.

The following description describes an example in which, as shown in FIG. 29, the ultrasonic transducer 77A corresponding to the attachment position where the distance from the reference point is Da2 is classified into group A, the ultrasonic transducer 77B corresponding to the attachment position where the distance from the reference point is Db2 (Db2>Da2) is classified into group B, the ultrasonic transducer 77C corresponding to the attachment position whose distance from the reference point is Dc2 (Dc2>Db2) is classified into group C, and the ultrasonic transducer 77D corresponding to the attachment position where the distance from the reference point is Dd2 (Dd2>Dc2) is classified into group D. In this example, the ultrasonic transducers 77 belonging to groups A and C are attached at a different distance from the main face 78S with respect to the direction perpendicular to the main face 78S than the ultrasonic transducers 77 belonging to groups B and D. In addition, the ultrasonic transducers 77 belonging to groups C and D are driven in opposite phase compared to those in group A and group B. For example, the ultrasonic transducers 77 can be driven in opposite phase by changing connections of the input terminals that supply the drive signal.

The attachment position with respect to the direction orthogonal to the main face 78S can be adjusted by the attachment 79 shown in FIG. 31. On the main face 78S of the bottom support 78, the attachment 79 shown in FIG. 31 is attached. Attachment 79 is configured to adjust the distance between the ultrasonic transducers 77 belonging to groups B and D and the main face 78S. Specifically, the attachment has an substantial uniform thickness and provides an substantially planar area above the main face 78S that is spaced apart by a distance corresponding to the thickness with respect to a direction perpendicular to the main face 78S.

Ultrasonic transducers 77 belonging to group A or group C are directly attached to the mount. On the other hand, the ultrasonic transducers 77 belonging to groups B and D are attached to the mount via attachment 79. Therefore, the distance from each ultrasonic transducer 77 to the main face 78S is not uniform. In other words, each ultrasonic transducer 77 is attached to the bottom support 78 at a different distance from the main face 78 than at least one of the other ultrasonic transducers 77.

As an example, each of the plurality of ultrasonic transducers 77 belonging to group A or group B (i.e., ultrasonic transducers without inverted drive signal input 77) is arranged at an offset position such that the greater the distance from a reference point on the main face 78S to the mount corresponding to the ultrasonic transducer 77, the greater the distance from the main face 78S with respect to a direction perpendicular to the main face 78S. Specifically, the attachment position of each ultrasonic transducer 77 belonging to group A or group B can be determined as a position on a sphere centered at a point a predetermined distance away from the main face 78S (corresponding to the target point when the bottom support 78 is mounted on the base 71, hereinafter referred to as “the designed focal point of the ultrasonic transducer 77”). In other words, the attachment positions of the plurality of ultrasonic transducers 77 belonging to group A or group B can be determined so that the distance from the designed focal point is substantially equal to each other. The same applies to the attachment positions of multiple ultrasonic transducers 77 belonging to group C or group D (i.e., ultrasonic transducers 77 with inverted drive signal input).

It should be noted that the ultrasonic transducers 77 belonging to group C or group D are driven in opposite phase compared to those belonging to group A or group B. Therefore, the distance from the attachment position of the ultrasonic transducer 77 belonging to group C or group D to the designed focal point of the ultrasonic transducers 77 is different from the distance from the attachment positions of the plurality of ultrasonic transducers 77 belonging to group A or group B to the designed focal point of the ultrasonic transducers 77.

Specifically, as explained in Modification 3, the distance from the designed focal point of the ultrasonic transducers 77 to the ultrasonic transducer 77 belonging to group C or group D is about half a wavelength of the ultrasonic wave longer than the distance from the designed focal point of the ultrasonic transducers 77 to the ultrasonic transducers 77 belonging to group A or group B. In this case, ultrasonic waves radiated from the ultrasonic transducers 77 belonging to any group are in-phase and strengthen each other at the designed focal point of the ultrasonic transducers 77.

The bottom support 78 supports the connector on the main face 78S. The connector is connected to an external device (e.g., controller 30) to enable electrical signals to be exchanged.

With the ultrasonic transducer 77 attached (directly or indirectly via attachments) to the mount, the ultrasonic transducer 77 can maintain its position and orientation (i.e., remain stationary) with respect to the bottom support 78. The ultrasonic transducer 77 is electrically connected to the connector while attached to the mounts. The ultrasonic transducer 77 is configured to receive, via a connector, a drive signal transmitted by the controller 30 and to radiate, in accordance with the drive signal, an ultrasonic wave traveling along the second direction. The second direction is a different direction from the first direction described above, for example, the direction normal to the main face 78S of the bottom support 78. The ultrasonic waves radiated from ultrasonic transducers 77 converge at the focal point FP, which corresponds to the aforementioned designed focal point, as shown in FIG. 27. In other words, the side support 72, ultrasonic transducer 76, ultrasonic transducer 77, and bottom support 78 are configured such that the first focal point where ultrasonic waves radiated by ultrasonic transducers 76 converge coincide with the second focal point where ultrasonic waves radiated by ultrasonic transducers 77.

### (6-6-2) Summary

As explained above, the ultrasonic radiation apparatus 70 of Modification 6 has a side support 72, a plurality of ultrasonic transducers 76, a bottom support 78, and a plurality of ultrasonic transducers 77, and they are arranged such that the first focal point at which ultrasonic waves radiated by the ultrasonic transducers 76 converge coincide with the second focal point at which ultrasonic waves radiated by the ultrasonic transducers 77 converge. This allows the acoustic radiation pressure to be applied to the treatment area of the subject in a non-contact manner by aligning the treatment area of the subject with the target point. Therefore, the application of the ultrasonic radiation apparatus 70 of Modification 6 to a hair care apparatus can reduce adverse effects on hygiene in hair care. In addition, the ultrasonic radiation apparatus 70 has a bottom support 78 to which the multiple ultrasonic transducers 77 can be attached instead of converging the Z-directional end to a single apex with, resulting in highly efficient placement of the ultrasonic transducers. In other words, according to the ultrasonic radiation apparatus 70, the required acoustic radiation pressure can be achieved while suppressing the dimension in the Z direction.

In the ultrasonic radiation apparatus 70, the plurality of ultrasonic transducers 77 are classified into groups based on the distance from the reference point which is the intersection of the perpendicular line descended from the designed focal point to the main face 78S of the bottom support 78 to the attachment position of the ultrasonic transducer 77. The ultrasonic transducer 77 belonging to the first group of the plurality of groups may be driven in opposite phase compared to the ultrasonic transducer 77 belonging to the second group of the plurality of groups. This allows the ultrasonic waves radiated from the plurality of ultrasonic transducers 77 to converge at the designed focal point while suppressing the adjustment range of the attachment position with respect to the direction perpendicular to the main face 78S for the plurality of ultrasonic transducers 77 attached at different distances from the reference point.

In the ultrasonic radiation apparatus 70, the plurality of ultrasonic transducers 77 are classified into groups based on the distance from the reference point which is the intersection of the perpendicular line descended from the designed focal point to the main face 78S of the bottom support 78 to the attachment position of the ultrasonic transducer 77. The ultrasonic transducer 77 belonging to the first group of the plurality of groups is attached to the main face 78S such that the distance from the main face 78S is different with respect to the direction orthogonal to the main face 78S compared to the ultrasonic transducer 77 belonging to the second group of the plurality of groups. This allows the attachment positions of the plurality of ultrasonic transducers 77, which are attached at different distances from the reference point, to be adjusted with respect to the direction orthogonal to the main face 78S and the distances from the designed focal point to the ultrasonic transducers 77 to be aligned. Thus, the ultrasonic waves radiated from the plurality of ultrasonic transducers 77 are to be converged at the focal point.

### (7) Other Modifications

The storage device 31 may be connected to the controller 30 via a network. In the above explanation, we have shown an example in which attachment 13 is attached on the main face 12S of the transducer support 12 to form a stepped staircase-like area on the main face 12S with respect to the direction orthogonal to the main face 12S.

However, at least part of attachment 13 may be attached to ultrasonic transducer 16.

Alternatively, the main face 12S of the transducer support 12 may be configured in a three-dimensional shape. As an example, the main face 12S may be modified to include a first region and a second region. The first region is a substantially planar face. The second region is offset from the first region in a direction orthogonal to the first region toward the direction in which the ultrasonic waves travel, and is staircase-shaped with steps in that direction. At least one of the plurality of mounts 14 is provided in the second region. This eliminates the need for attachments 13 or reduces the number of attachments 13.

Base 11 may be attached to a movable mechanism. The movable mechanism is configured to change at least one of the positions or postures of the movable mechanism. This makes it possible to adjust the position of the ultrasound focal point because the position or posture of the base 11 is also changed in response to changes in the position or posture of the movable mechanism.

The above description shows an example where the number of transducer supports 12 provided by the ultrasonic radiation apparatus 10 is five or six. However, the number of transducer supports 12 provided by the ultrasonic radiation apparatus 10 may be between two and four, or may be seven or more.

In particular, in the above description, examples are shown in which the five or six transducer supports 12 are arranged so that their main faces 12S of them correspond to five or six equilateral triangle sharing a vertex, the triangles constituting a regular icosahedron. The arrangement of multiple transducer support 12 is not limited to these examples, but such an arrangement is highly scalable. In short, suppose we add any number of transducer supports 12 with main faces 12S corresponding to any of the equilateral triangles that constitute the regular icosahedron. Since the normals of the equilateral triangles constituting the regular icosahedron all pass through the center of the icosahedron, the ultrasonic waves radiated by the multiple ultrasonic transducers 16 attached to the added transducer support 12 can be reliably focused to the target point.

The above explanation shows an example where the number of ultrasonic transducers 16 mounted on the transducer support 12 is 10. However, the number of ultrasonic transducers 16 mounted on the transducer support 12 may be no more than 9 or may be 11.

In the above explanation, each ultrasonic transducer 16 is attached so that the distance from the main surface 12S of the transducer support 12 to the transducer 16 can be adjusted in three steps. However, each ultrasonic transducer 16 may be attached so that the distance from the main face 12S of the transducer support 12 to the transducer 16 can be adjusted in two, four or more steps.

The hair care apparatus 1 according to the present embodiment or any modification may be further equipped with a light emission element. The light emission element is, for example, an LED. The light emission elements is located in the ultrasonic radiation apparatus 10. The light emission element is driven by the controller 30 to emit a ray of light traveling along the direction of ultrasonic radiation (Z+ direction), for example. This allows the affected area to be irradiated with a light ray in addition to ultrasonic waves for further hair growth or regrowth.

The hair care apparatus 1 according to the present embodiment or any modification may be further equipped with an electric needle. The electric needle is installed so that it protrudes in the radiation direction (Z+ direction). As a first example, the electric needle constitutes part or all of the annular wall 42a of the hood 42 of the hair care apparatus 1 in FIGS. 14 and 15. As a second example, the electric needle constitutes part or all of the guide 1d of the hair care apparatus 1 in FIGS. 12A and 12B. As a third example, the electric needle is installed independently of (e.g., as part of the head 41 configuration) the hood 42 of the hair care apparatus 1 in FIGS. 14 and 15. As a fourth example, the electric needle is installed independently of the guide 1d of the hair care apparatus 1 in FIGS. 12A and 12B. As a fifth example, the electric needle is configured as a head attachment for the hair care apparatus 1 shown in FIGS. 14 and 15.

The electric needle is energized under the control of the controller 30 and applies electrical stimulation to the human head while in contact with the head. This provides electrical stimulation around the affected area for further hair regrowth or hair growth.

The fifth example of the external shape of the hair care apparatus 1 can be modified to have a movable part. The movable part is mounted on the surface of the housing 50 that faces the human head while the housing 50 is attached to the human head (hereinafter referred to as the “inner surface”). The movable part allows the ultrasonic radiation apparatus 10 to move over the inner surface of the housing 50. According to this modification, the user can perform hair care while wearing the hair care apparatus 1 on the head, similar to the fifth example of the external shape of the hair care apparatus 1. This means that the user can use both hands freely while performing hair care. Compared to the fifth example of the external shape of the hair care apparatus 1, the number of device attachment sections 51 can be reduced (e.g., to one).

Although an example of a hair care apparatus 1 is shown in this embodiment, the scope of application of this embodiment is not limited to this. This embodiment is also applicable to apparatuses that are expected to be improved by non-contact vibration pressure stimulation by ultrasound (e.g., apparatus to care for wounds on the skin or apparatus to care for the skin (beauty apparatus, as an example)).

Modification 6 describes an example of grouping multiple ultrasonic transducers (ultrasonic transducers 76 or 77) based on their distance from a reference point. From the viewpoint of maximizing the ultrasonic sound pressure at the focal point, it is preferable to classify only ultrasonic transducers with matching distances from the reference point into the same group. On the other hand, if a decrease in ultrasonic sound pressure at the focal point can be tolerated, ultrasonic transducers whose distance from the reference point is within a predetermined numerical range may be classified into the same group.

When the difference between the first and second group in the ideal attachment position of the ultrasonic transducers with respect to the direction orthogonal to the main face of the transducer support (side support 72 or bottom support 78) is within a predetermined numerical range, for example, the attachment position of the first group may be aligned with that of the second group. Here, the ideal attachment position refers to the attachment position where the distance from the designed focal point of the ultrasonic transducers is a predetermined value. By aligning the attachment position of the first group with that of the second group, the sound pressure of the ultrasonic waves at the focal point is reduced, but the required number of variations in the attachment position of the ultrasonic transducer with respect to the direction orthogonal to the main face is reduced compared to the number of groups. This reduces the number of steps required for the attachment and simplifies the structure of the attachment.

In Modification 6, a plurality of ultrasonic transducers are grouped according to the distance from the reference point to the attachment position of the ultrasonic transducer, and at least one of the following is different among the groups: the attachment position of the ultrasonic transducer in the direction orthogonal to the main face and whether the drive signal is inverted. However, when a plurality of ultrasonic transducers are classified into two groups, the attachment position of the ultrasonic transducers with respect to the direction orthogonal to the main face may be the same regardless of the group, and only whether the drive signal is inverted input or not may be different.

According to the above disclosure, adverse effects on hygiene in hair care can be reduced.

Although the embodiments of the present invention are described in detail above, the scope of the present invention is not limited to the above embodiments. Further, various modifications and changes can be made to the above embodiments without departing from the spirit of the present invention. In addition, the above embodiments and modifications can be combined.

### REFERENCE SIGNS LIST

1 : Hair care apparatus

1a: Base cover

1b: Grip

1c: Housing

1d: Guide

1e: First arm

1f: Second arm

1g: Pedestal

10 : Ultrasonic radiation apparatus

11 : Base

12 : Transducer support

12S : Main face

13 : Attachment

14 : Mount

15 : Connector

16 : Ultrasonic transducer

30 : Controller

31: Storage device

32 : Processor

33: Input/output interface

34 : Communication Interface

41 : Head

42 : Hood

43 : Grip

44 : Power button

45 : Mode switch

46 : Jack

47 : Lamp

48 : Head attachment

50 : Housing

51 : Device attachment section

60 : Notification device

61 : Head

62 : Grip

63 : Blower

64 : Heater

65 : Nozzle

66 : Cartridge

67 : Vibration motor

70 : Ultrasonic radiation apparatus

71 : Base

72 : Side support

76 : Ultrasonic transducer

77 : Ultrasonic transducer

78 : Bottom support

79 : Attachment

What is claimed is:

1. An ultrasonic radiation apparatus comprising:
a first transducer support with a substantially planar first main face, the first transducer support having a plurality of first mounts on the first main face;
a second transducer support with a substantially planar second main face, the second transducer support having a plurality of second mounts on the second main face and being arranged such that a second normal of the second main face intersects a first normal of the first main face at a common target point;
a plurality of first ultrasonic transducers attached to the plurality of first mounts to radiate ultrasonic waves traveling along the first normal; and
a plurality of second ultrasonic transducers attached to the plurality of second mounts to radiate ultrasonic waves traveling along the second normal.

2. The apparatus according to claim 1, wherein
each of the plurality of first ultrasonic transducers is attached at an offset position such that the greater a distance from a reference point on the first main face to the first mount corresponding to the first ultrasonic transducer, the greater a distance from the first main face in a direction perpendicular to the first main face.

3. The apparatus according to claim 2, wherein
attachment positions of the plurality of first ultrasonic transducers are determined so that distances from a point a predetermined distance away from the first main face are substantially equal to each other.

4. The apparatus according to claim 1, wherein
the first transducer support has an attachment on the first main face,
the attachment forms a stepped staircase-like region with respect to a direction perpendicular to the first main face, and
at least one of the plurality of first ultrasonic transducers is attached via the attachment to the first mount where the first ultrasonic transducer is attached.

5. The apparatus according to claim 1, wherein
the first transducer support and second transducer support are arranged such that a distance from the first main face to the target point and a distance from the second main face to the target point are substantially equal to each other.

6. An ultrasonic radiation apparatus comprising:
a first transducer support with a substantially planar first main face, having a plurality of first mounts on the first main face;
a second transducer support with a substantially planar second main face, having a plurality of second mounts on the second main face;
a plurality of first ultrasonic transducers attached to the plurality of first mounts to radiate ultrasonic waves traveling along a first direction; and
a plurality of second ultrasonic transducers attached to the plurality of second mounts to radiate ultrasonic waves traveling along a second direction different from the first direction, and wherein
the first transducer support, the plurality of first ultrasonic transducers, the second transducer support, and the plurality of second ultrasonic transducers are arranged such that a first focal point at which ultrasonic waves radiated by the plurality of first ultrasonic transducers converge coincides with a second focal point at which ultrasonic waves radiated by the plurality of second ultrasonic transducers converge.

7. The apparatus according to claim 6, wherein
the plurality of second ultrasonic transducers are classified into a plurality of groups based on a distance from a reference point, which is an intersection of a perpendicular line descending from the second focal point to the second main face and the second main face, to an attachment position of the second ultrasonic transducer, and
the second ultrasonic transducer belonging to a first group of the plurality of groups is driven in opposite phase compared to the second ultrasonic transducer belonging to a second group of the plurality of groups.

8. The apparatus according to claim 6, wherein
the plurality of second ultrasonic transducers are classified into a plurality of groups based on a distance from a reference point, which is an intersection of a perpendicular line descending from the second focal point to the second main face and the second main face, to an attachment position of the second ultrasonic transducer, and
the second ultrasonic transducer belonging to a first group of the plurality of groups is attached to the second main face such that a distance from the second main face is different with respect to a direction orthogonal to the second main face compared to the second ultrasonic transducer belonging to a second group of the plurality of groups.

9. A hair care apparatus comprising:
an ultrasonic radiation apparatus according to claim 1;
a controller that drives the plurality of first ultrasonic transducers and the plurality of second ultrasonic transducers.

10. The apparatus according to claim 9, further comprising:
a head that supports the first transducer support and second transducer support; and
a hood that is attached to the head and has an annular wall protruding in a radiation direction, which is a common direction of travel of ultrasonic waves radiated by the plurality of first ultrasonic transducers and second ultrasonic transducers, and wherein
the target point is included in a tip surface through a plurality of tips of the annular wall of the hood that protrude the most in the radiation direction.

11. The apparatus according to claim 10, wherein
the annular wall of the hood periodically increases or decreases in dimension in the radiation direction depending on a position in a plane perpendicular to the radiation direction.

12. The apparatus according to claim 10, wherein the hood is removable from the head.

13. The apparatus according to claim 10, further comprising a grip that supports the head and extends in a direction closer to the radiation direction than in a direction orthogonal to the radiation direction.

14. The apparatus according to claim 9, further comprising an electric needle protruding in a radiation direction, which is a common direction of travel of the ultrasonic waves radiated by the plurality of first ultrasonic transducers and the plurality of second ultrasonic transducers.

15. The apparatus according to claim 9, further comprising a housing that has a plurality of device attachment sections at a plurality of different positions on a surface opposite to a human head when the housing is worn on the human head, the ultrasonic radiation apparatus being attachable to and detachable from the plurality of device attachment sections.

16. The apparatus according to claim 9, further comprising:
a housing that is able to be worn on a human head; and
a movable part that enables the ultrasonic radiation apparatus to move across a surface of the housing that is opposite the human head when the housing is worn on the human head.

17. The apparatus according to claim 9, further comprising a light emission element that emit a light ray traveling along a radiation direction, which is a common direction of travel of the ultrasonic waves radiated by the plurality of first ultrasonic transducers and the plurality of second ultrasonic transducers.

18. The apparatus according to claim 9, further comprising notification module that ultrasonic waves are being radiated by the ultrasonic radiation apparatus.

19. The apparatus according to claim 9, wherein the first ultrasonic transducer farthest from a reference point on the first main face is driven in opposite phase to the first ultrasonic transducer closest to the reference point.

20. A hair care apparatus comprising:
a plurality of side supports;
a plurality of first ultrasonic transducers attached to each of the plurality of side supports;
a bottom support;
a plurality of second ultrasonic transducers attached to the bottom support; and
a controller that substantially simultaneously drives the plurality of first ultrasonic transducers and the plurality of second ultrasonic transducers, and wherein
each of the plurality of side supports has a main face,
the bottom support has a main face,
the bottom support and the plurality of side supports are arranged so that the main face of the bottom support and the main faces of the plurality of side supports are non-parallel to each other,
the plurality of side supports are arranged so that normals extending from the main faces of the plurality of side supports toward the direction of ultrasonic radiation by the plurality of first ultrasonic transducers does not intersect the main face of the bottom support, and
the plurality of side supports, the plurality of first ultrasonic transducers, the bottom support, and the plurality of second ultrasonic transducers are arranged such that a first focal point at which ultrasonic waves radiated by the plurality of first ultrasonic transducers converge coincides with a second focal point at which ultrasonic waves radiated by the plurality of second ultrasonic transducers converge.
